SEQUENCE LISTING (24 sequences)

SEQ ID NO: 1 - protein, 154 aa, Homo sapiens [sequence omitted]

SEQ ID NO: 2 - protein, 117 aa, synthetic construct
QVQLVESGGGWVQTGGSLKLSCVVSGINFADSRMGWYRQAPGNQYDPIAEMNVGGLRKYADSVKTRFTISRNNVKNTVYLQMDSLKPEDTGVYVCGAETIWDSARYWGQGIQVTVSS

SEQ ID NO: 3 - protein, 116 aa, synthetic construct
HVQLVESGGGSVQAGGSLRLSCVVSGINFGDSAAGWYRQVPGQLREFVASLSRSGFRNFADSVKDRFSMSRVNAKNTVFLQMNDLKVEDTAVYYCNVGDTLPSQYWGQGTQVTVSS

SEQ ID NO: 4 - protein, 116 aa, synthetic construct
AVQLVDSGGGSVQAGGSLRLSCVVSGIDFGDSAAGWYRQVPGQLREFVASLSRSGFRNFADSVKDRFSMSRVNAKNTVFLQMNDLKVEDTAVYYCNVGDTLPSQYWGQGTQVTVSS

SEQ ID NO: 5 - protein, 116 aa, synthetic construct
EVQLVESGGGSVQAGGSLRLSCVVSGINFADSRMGWYRQAPGQLREFVASLSRSGFRNFADSVKDRFSMSRVNAKNTVFLQMNDLKVEDTAVYYCNVGDTLPSQYWGQGTQVTVSS

SEQ ID NO: 6 - protein, 124 aa, synthetic construct
QVQLVESGGGWVQAGGSLRLSCVVSGTNFNDRSMGWYRQAPGKQRELVATMSFGGRRNYVDAVKARFTISRDNRKNTTYLLMNDLEPDDTAVYYCAAGHVYASVAPATTWIEYWGQGTQVTVSS

SEQ ID NO: 7 - protein, 124 aa, synthetic construct
QVQLVESGGGWVQAGGSLRLFCVVSGTNFNDRSMGWYRQAPGKERELVATMSYGGRRNYADAVKARFTISRDNRKNTTYLLMNDLEPDDTAVYYCAAGHVLESVVPATTWIEYWGQGTQVTVSS

SEQ ID NO: 8 - protein, 124 aa, synthetic construct
QVQLVESGGGWVQAGGSLRLSCVVSGTNFNDRSMGWYRQAPGKERELVATLSYGGRRNYVDAVKARFTMSRDNRKNTTYLLMNDLEPDDTAVYYCAAGHVFESVVPATTWIEYWGQGTQVTVSS

SEQ ID NO: 9 - protein, 124 aa, synthetic construct
QVQLVESGGGWVQAGGSLRLSCVVSGTNFNDRSMGWYRQAPGKERELVATLSYGGRRNYVDAVKARFTMSRDNRKNTTYLLMNDLEPDDTAVYYCAAGHVLESVVPATTWIEYWGQGTQVTVSS

SEQ ID NO: 10 - protein, 123 aa, synthetic construct
QVQLVESGGGSVQAGGSLRLSCAHSGSNFNDRSMGWYRQAPGKERELVATLSYGGRRNYVDAVKARFSMSRDNRKNTTYLLMNDLEPDDTAVYYCAAGHVLESVVPATTWIEYWGQGTQVTVS

SEQ ID NO: 11 - protein, 116 aa, synthetic construct
QVQLVESGGGLVQAGGSLRLSCAASGIPFNEKIMGWYRQAPGNERELVAYIDTTSRTWHKDYLRDRFTISRDNAKNAAYLQMNSLKPEDTAVYYCATLWNTPTNYWGKGTQVTVSS

SEQ ID NO: 12 - protein, 116 aa, synthetic construct
QVQLVESGGGLVQAGGSLRLSCAASGIPFNEKIMGWYRQAPGNERELVAYIDTTSRTWHKDYLRDRFTISRDNAENAAYLQMNSLKPEDTAVYYCATLWNTPTNYWGKGTQVTVSS

SEQ ID NO: 13 - protein, 111 aa, synthetic construct
QVKLEESGGGLVQAGESRRLSCAPSGRIFGINGMGWYRRTPGKQRETVAILYSDGTITYGDSVKGRFTISRDNANGTPSLQMTDLKPEDAGLYICNVGGHWGSGTQVTVSS

SEQ ID NO: 14 - protein, 116 aa, synthetic construct
QVQLVESGGGLVQAGGSLRLSCAASGSVNVMGWYRQAPGRERTLVAHITSGSTTTYVDSVKGRFTISRDNAKNTVYLQMNSLKPEDTAVYYCAAEGAFTGWGPEYWGQGTQVTVSS

SEQ ID NO: 15 - protein, 116 aa, synthetic construct; identical to SEQ ID NO: 14

SEQ ID NO: 16 - protein, 119 aa, synthetic construct
QVKLEESGGGLVQAGGSLRLACTASGRIRNINAMGWYRQAPGRERTLVAHITSGSTTTYVDSVKGRFTISRDNAKNTVYLQMNSLKPEDTAVYYCAAEGAFTGWGPEYWGQGTQVTVSS

SEQ ID NO: 17 - protein, 119 aa, synthetic construct
QVQLVESGGGLVQPGGSLRLSCAASGSDAILNVMGWYRQAPGWERTLVGHITSGGTTTYVDAVKGRFTISRDNAENTVYLQMNSLKPEDTAVYYCAAEGAFTGWGPDYWGQGTQVTVSS

SEQ ID NO: 18 - protein, 119 aa, synthetic construct
QVQLVESGGGLVQPGGSLRLSCAASGSDAILNVMGWYRQAPGWERTLVGHITSGGTTTYVDAVKGRFTISRDNAKNTVYLQMNSLKPEDTAAYYCAAEGAFTGWGPDYWGQGTQVTVSS

SEQ ID NO: 19 - protein, 119 aa, synthetic construct
QVQLVESGGGLVEAGGSLRLSCAASGSDAILNVMGWYRQAPGWERTLVGHITSGGTTTYVDAVKGRFTISRDNAKNTVYLQMNSLKPEDTAVYYCAAEGAFTGWGPDYWGQGTQVTVSS

SEQ ID NO: 20 - protein, 119 aa, synthetic construct
QVQLVESGGGLVQAGGSLRLSCIASGSFLSINVMGWYRQAPGKQRELVGHITKGGTTTYTDSVKGRFTISRDNAKNTVYLQMNSLKLEDTAVYYCAAEGAFTGWPPEYWGQGTQVTVSS

SEQ ID NO: 21 - protein, 118 aa, synthetic construct
QVKLEESGGGLVQAGGSLRLSCIASGSFLSINVMGWYRQAPGKQRELVGHITKGVTTTYTDSVKGRFTISRDNAKNTVYLQMNSLKLEDTAVYYCAAEGAFTGWPPEYWGQGTQVTVS

SEQ ID NO: 22 - protein, 117 aa, synthetic construct
QVQLVESGGGLVQAGGSLRLSCVASGSYPNVMGWYRQAPGKQRLLVAHITSGGTTTYADSVKGRFTISRDNAKNTVYLQMNSLKLEDTAVYYCAAEGAFTGWGPEFWGQGTQVTVSS

SEQ ID NO: 23 - protein, 117 aa, synthetic construct
QVQLVESGGGLVQPGGSLRLSCAASGSYPNVMGWYRQAPGKQRLLVAHITSGGTTTYADSVKGRFTISRDNAQNTVYLQMNSLKLEDTAVYYCAAEGAFTGWGPEFWGQGTQVTVSS

SEQ ID NO: 24 - protein, 119 aa, synthetic construct
QVQLVESGGGLVQAGESLRLSCAASGSNLDINVMGWYRQAPGKQRLLVAHITRGGSTTYADSVKGRFTISRDNDKNTVYLQMNSLKPEDTAVYYCAAEGAFTGWGPEYWGQGTQVTVSS

(19) United States
(12) Patent Application Publication
(10) Pub. No.: US 2025/0388697 A1
(43) Pub. Date: Dec. 25, 2025

(54) ANTI-SOD1 NANOBODIES

(71) Applicant: University of Massachusetts, Westborough, MA (US)

(72) Inventors: Daryl A. Bosco, Boylston, MA (US); Miguel Sena Esteves, Westford, MA (US); Meenakshi Sundaram Kumar, Worcester, MA (US); Megan E. Fowler-Magaw, Sterling, MA (US)

(21) Appl. No.: 18/877,663

(22) PCT Filed: Jun. 21, 2023
(86) PCT No.: PCT/US2023/025868

§ 371 Date: Dec. 20, 2024

Related U.S. Application Data
(60) Provisional application No. 63/354,385, filed on Jun. 22, 2022.

(51) Int. Cl. C07K 16/40; A61K 38/18; A61K 39/00; A61K 48/00; A61P 25/28; G01N 33/573

(52) U.S. Cl. CPC C07K 16/40; A61K 38/185; A61K 48/005; A61K 48/0075; A61P 25/28; G01N 33/573; A61K 2039/54; C07K 2317/569; C07K 2319/95; G01N 2333/90283

(57) ABSTRACT

Composition and methods of diagnosing, monitoring, and treating subjects with a motor neuron pathology, such as motor neuron disorders (including but not limited to amyotrophic lateral sclerosis (ALS)) and neuropathies.

### CROSS-REFERENCE TO RELATED APPLICATIONS

This application claims the benefit of priority to U.S. Application No. 63/354,385, filed on Jun. 22, 2022, the contents of which are hereby incorporated by reference.

### TECHNICAL FIELD

Described herein are compositions and methods for diagnosing, monitoring, and treating subjects with motor neuron pathology such as motor neuron disorders (including but not limited to amyotrophic lateral sclerosis (ALS), related neurodegenerative diseases (including but not limited to Parkinson's Disease) and neuropathies, based on imaging or delivery of labeled or unlabeled mis-SOD1 nanobodies.

### BACKGROUND

Accumulation of misfolded protein is a hallmark feature of most neurodegenerative diseases including amyotrophic lateral sclerosis (ALS) and Alzheimer's, Parkinson's and Huntington's diseases (AD, PD and HD, respectively). In this context, the term “misfold” refers to a protein adopting a pathological conformation. Misfolded proteins generally accumulate into aggregates, which are often composed of other proteins and cellular factors. The different misfolded species (e.g., soluble misfolded precursors, intermediate protofibrils and end-stage insoluble aggregates) that populate the protein aggregation pathway all exert some degree of toxicity, albeit to different degrees, and ultimately induce a detrimental disturbance of proteostasis (the balance between protein production and turnover). Targeting proteins that misfold, either at the RNA or protein level, represents a promising therapeutic strategy across multiple neurodegenerative disorders.

Dominant mutations in the gene encoding Cu/Zn superoxide dismutase 1 (SOD1) account for 20-25% of the inherited forms of amyotrophic lateral sclerosis (ALS) (1). Normally SOD1 is present in both the cytoplasm and the nucleus (2). As a key cytosolic anti-oxidizing enzyme, SOD1 catalyzes conversion of harmful superoxide radicals to hydrogen peroxide and oxygen. SOD1 also functions as a transcription factor in the nucleus, where it regulates genome stability and DNA damage repair to mitigate oxidative stress (3).

Multiple lines of evidence indicate that mutations in SOD1 cause ALS primarily through a gain of toxic function. For instance, overexpression of ALS-linked mutant SOD1 in rodent models recapitulates ALS phenotypes, including motor neuron degeneration and neuroinflammation (4). The toxicity of ALS-linked SOD1 may arise from mutation-induced structural perturbations in SOD1 resulting in toxic, misfolded conformations (5,6). Indeed, ALS-linked SOD1 variants adopt a misfolded and thermodynamically destabilized conformation that correlates with severity of the human disease (7). Misfolding of SOD1 can also be initiated by aberrant post-translational modifications, which are relevant in cases of ALS without SOD1 mutation (5,8-11).

To overcome the toxicity of ALS-linked SOD1, gene-silencing of SOD1 via microRNAs (12) and antisense oligonucleotides (ASOs) (13,14) has been proposed. However, there may be adverse consequences when these therapeutics also reduce expression of wild-type (WT) SOD1 (5,15). Indeed, knockout of SOD1 in mice results in defective axonal homeostasis and stress response, as well as oxidative damage within the nucleus (16-19). Misfolded and mutant SOD1 variants also accumulate in the cytoplasm (11,20-23), which could lead to a loss of nuclear SOD1 activity in a manner that also contributes to ALS and other neurodegenerative disorders (24,25).

Immunotherapy that selectively targets misfolded SOD1 species, while not reducing expression of SOD1 WT, may represent an alternative and viable therapeutic approach. Active immunization using misfolded SOD1 antigens or passive immunization with antibodies specific to misfolded SOD1 improved survival in transgenic rodent models expressing ALS-linked SOD1 mutations (26-30). However, the use of conventional monoclonal antibody-based therapy is limited by insufficient pharmacokinetics, high production costs and a general inefficacy of antibodies for entering cells (31). Engineered antibody fragments overcome some of these shortcomings. In ALS mouse models, beneficial effects have been observed using anti-SOD1 single chain variable fragments (scFvs) that consist of only the variable domains of the immunoglobulin heavy and light chains (32,33). Recently, a smaller and more versatile antibody format comprised of a single antigen-binding domain has emerged. These so-called “nanobodies” are derived from the variable domain of heavy-chain alone antibodies found in camelid sera (34). With a molecular weight of ˜15 kDa, nanobodies are produced efficiently and in high yields as recombinant proteins (34). Nanobodies are also more effectively delivered into cells through gene therapy approaches as compared to conventional antibodies (31). Nanobodies can be further engineered to enhance their performance, such as engaging cellular protein degradation machinery (35) and bypassing the blood-brain barrier (36). In the context of neurological disorders, nanobodies have already shown promise in preclinical models of Parkinson's (35) and Alzheimer's disease (37). However, reports of nanobodies targeting SOD1 remain limited.

### SUMMARY

Provided herein are single-domain antibodies that bind to Superoxide dismutase 1 (SOD1) comprising an amino acid sequence that is at least 80% identical to of any one of SEQ ID NO: 2-24. In some embodiments, SOD1 is human SOD1. In some embodiments, the single-domain antibodies described herein bind to mutant SOD1. In some embodiments, the mutant SOD1 is characterized by a mutation of amino acids at positions 4 and/or 93. In some embodiments, the single-domain antibody binds both wild-type and mutant SOD1.

Also provided herein are fusion proteins comprising a single-domain antibody as described above, fused to a tag. In some embodiments, the tag is a degradation tag. In some embodiments, the degradation tag is PEST.

Also described herein are nucleic acid molecules encoding the single-domain antibodies described above. In some embodiments, provided herein are vectors comprising the nucleic acid molecules. In other embodiments, provided herein are host cells comprising the nucleic acid molecules.

Also provided herein are methods of diagnosing and monitoring a motor neuron pathology the method comprising: administering a single-domain antibody as described above to a subject.

Also provided herein are methods of treating a motor neuron pathology or improving symptoms of the motor neuron pathology in a subject, the method comprising: administering a therapeutically effective amount of a single-domain antibody as described above to the subject.

In any of the methods, in some embodiments, the motor neuron pathology is amyotrophic lateral sclerosis (ALS). In any of the methods, in some embodiments, the single-domain antibody is administered intracerebroventricularly. In any of the methods, in some embodiments, the single-domain antibody is administered through gene therapy. In any of the methods, in some embodiments, the method further comprises administering a neurotrophin.

Also described herein are medicaments comprising: a single-domain antibody as described above and a pharmaceutically acceptable excipient.

### Definitions

The term “antibody” is used herein in the broadest sense and encompasses various antibody structures, including but not limited to, an immunoglobulin molecule that recognizes and binds a target through at least one antigen-binding site, polyclonal antibodies, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

An “antigen” is a molecule comprising at least one epitope. The antigen may for example be a polypeptide, nucleic acid, polysaccharide, protein, lipoprotein or glycoprotein.

A “complementarity determining region” or “CDR” is a hypervariable region of the antigen-binding region of an antibody. The CDRs are interspersed between regions that are more conserved, termed framework regions (FRs). The antigen-binding region of an antibody may thus comprise one or more CDRs and FRs, usually in each variable domain three CDRs and four FRs arranged from amino-terminus to carboxy-terminus in the following order: FR1, CDR1, FR2, CDR2, FR3, CDR3, FR4.

An “epitope” is a determinant capable of specific binding to an antibody. Epitopes may for example be comprised within polypeptides or proteins. Epitopes may be continuous or discontinuous, wherein a discontinuous epitope is a conformational epitope on an antigen which is formed from at least two separate regions in the primary sequence of the protein, nucleic acid or polysaccharide.

The term “antibody fragment” as used herein refers to a molecule other than an intact antibody that comprises a portion of an antibody and generally an antigen-binding site. Examples of antibody fragments include, but are not limited to, Fab, Fab′, F(ab′)2, Fv, single chain antibody molecules, scFv, sc(Fv)₂, disulfide-linked scFv (dsscFv), diabodies, tribodies, tetrabodies, minibodies, dual variable domain antibodies (DVD), single variable domain antibodies (e.g., camelid antibodies or nanobodies), and multispecific antibodies formed from antigen-binding antibody fragments.

The present disclosure relates primarily to single variable domain antibodies, also referred to as “nanobody” herein. A single variable domain antibody is an antibody fragment consisting of a single monomeric variable antibody domain. Single variable domain antibodies comprise only one single domain or fragment of a domain of a whole antibody. The single domain may be a heavy chain constant region (CH), a heavy chain variable region (VH), a light chain constant region (CL) or a light chain variable region (VL) or a fragment thereof.

The term “inhibitory” as used in the phrase “inhibitory single-domain antibody” or “inhibitory nanobody” herein, refers to the fact that the nanobody can inhibit the function and/or activity of its target protein. In case of wild-type SOD1, this means that the superoxide dismutase activity is inhibited. In case of mutant SOD1 that has a gain of function, typically it is meant that the (toxic) new function is inhibited, although this may also mean that the enzymatic activity is inhibited, or inhibited as well. For instance, inhibition may result in decrease of aggregation of mutant SOD1. Importantly, inhibition or decrease in toxic function may also be evaluated as an increase of another parameter, e.g., the inhibition may be evaluated by an increase in axonal length or an extended life span. “Inhibitory” can mean full inhibition (no enzymatic activity and/or toxic effect is observable) or may mean partial inhibition. For instance, inhibition can mean 10% inhibition, 20% inhibition, 25% inhibition, 30% inhibition, 40% inhibition, or more. Particularly, inhibition will be at least 50%, e.g., 50% inhibition, 60% inhibition, 70% inhibition, 75% inhibition, 80% inhibition, 90% inhibition, 95% inhibition or more. Percentage of inhibition typically will be evaluated against a suitable control (e.g., treatment with an irrelevant nanobody, or a wild-type subject versus a diseased subject), as will be readily chosen by the skilled person.

“Affinity” refers to the strength of binding between receptors and their ligands, for example between an antibody and its antigen. The affinity of an antibody can be defined in terms of the dissociation constant, K_{D}, which is an equilibrium constant that measures the propensity of a molecular complex to separate (dissociate) reversibly into the molecules forming the complex. In one aspect, K_{D} is defined as the ratio k_{off}/kₒₙ, where k_{off} and kₒₙ are the rate constants for association and dissociation of the molecular complex. Preferably affinity is determined by calculating the dissociation constant K_{D} based on IC₅₀ values. Thus, the affinity is measured as an apparent affinity.

The term “humanized antibody” as used herein refers to an antibody or antibody fragment that comprises a human heavy chain variable region and a light chain variable region wherein the native CDR amino acid residues are replaced by residues from corresponding CDRs from a non-human antibody (e.g., mouse, rat, rabbit, or non-human primate), wherein the non-human antibody has the desired specificity, affinity, and/or activity.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of publicly available bioinformatic software tools. X-ray crystallography may be used to characterize an epitope on a target protein by analyzing the amino acid residue interactions of an antigen/antibody complex.

The term “specifically binds” as used herein refers to an agent that interacts more frequently, more rapidly, with greater duration, with greater affinity, or with some combination of the above to a particular antigen, epitope, protein, or target molecule than with alternative substances. A binding agent that specifically binds an antigen can be identified, for example, by immunoassays, ELISAs, surface plasmon resonance (SPR), or other techniques known to those of skill in the art. In some embodiments, an agent that specifically binds an antigen (e.g., human SOD1) can bind related antigens. Generally, a binding agent that specifically binds an antigen will bind the target antigen at a higher affinity than its affinity for a different antigen. The different antigen can be a related antigen. In some embodiments, a binding agent that specifically binds an antigen can bind the target antigen with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology in a Biacore system as described herein or as known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of an alanine for a valine is considered to be a conservative substitution. Methods of identifying nucleotide and amino acid conservative substitutions that do not eliminate binding are well-known in the art.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “motor neuron pathologies” refer to a class of neurological pathologies that are characterized by the progressive loss of the structure and function of motor neurons and motor neuronal cell death. Non-limiting examples of motor neuron pathologies include amyotrophic lateral sclerosis, progressive bulbar palsy, pseudobulbar palsy, primary lateral sclerosis, progressive muscular atrophy, spinal muscular atrophy, post-polio syndrome, diverse types of peripheral neuropathy and traumatic nerve injury. A health care professional may diagnose a subject as having a motor neuron pathology by the assessment of one or more symptoms in the subject. Non-limiting symptoms of a motor neuron pathology in a subject include difficulty lifting the front part of the foot and toes; difficulty lifting the whole leg or standing or walking; weakness in arms, legs, feet, or ankles; hand weakness or clumsiness; muscle cramps and atrophy; twitching in arms, shoulders, torso and legs; stiffness of movement of the arms and legs in some cases; weakness of the tongue and pharyngeal muscles leading to difficulty speaking, chewing and swallowing; and muscle paralysis. Alternatively, a health care professional may diagnose a subject as having a sensory or a combined sensori-motor neurodegenerative disorder by the assessment of one or more symptoms in the subject such as spontaneous muscle twitching; tingling or pain in parts of body; electric shock sensations that occur with head movements; tremor; unsteady gait; misinterpretation of spatial relationships; loss of automatic movements; impaired posture and balance; stiff muscles; bradykinesia; involuntary jerking or writhing movements (chorea); involuntary, sustained contracture of muscles (dystonia); lack of flexibility; and others known in the art. A health care professional may also base a diagnosis, in part, on the subject's family history of a motor neuron or neurodegenerative disorder. A health care professional may diagnose a subject as having a motor neuron or neurodegenerative disorder upon presentation of a subject to a health care facility (e.g., a clinic or a hospital). In some instances, a health care professional may diagnose a subject as having a motor neuron disorder while the subject is admitted in an assisted care facility. Typically, a physician diagnoses a motor neuron disorder in a subject after the presentation of one or more symptoms.

The term “subject” refers to any animal (e.g., a mammal), including, but not limited to, humans, non-human primates, canines, felines, rabbits, rodents, and the like.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent, and that is generally safe, non-toxic, and has no effect on the pharmacological activity of the therapeutic agent. In general, those of skill in the art and government agencies consider a pharmaceutically acceptable excipient, carrier, or adjuvant to be an inactive ingredient of any formulation or any pharmaceutical composition.

The term “pharmaceutical formulation” or “pharmaceutical composition” as used herein refers to a preparation that is in such form as to permit the biological activity of the agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “therapeutic effect” as used herein refers to the effect and/or ability of an agent to reduce and/or ameliorate the severity and/or duration of (i) a disease, disorder, or condition in a subject, and/or (ii) a symptom in a subject. The term also encompasses the ability of an agent to (i) reduce or ameliorate the advancement or progression of a given disease, disorder, or condition, (ii) reduce or ameliorate the recurrence, development, or onset of a given disease, disorder, or condition, and/or (iii) to improve or enhance the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to both (i) therapeutic measures that aim to cure, slow down, lessen symptoms of, and/or halt progression of a pathologic condition or disorder and (ii) prophylactic or preventative measures that aim to prevent or slow the development of a targeted pathologic condition or disorder. Thus, those in need of treatment include those already with the disorder, those at risk of having/developing the disorder, and those in whom the disorder is to be prevented.

The term “prevent” or “prevention” or “preventing” as used herein refers to the partial or total inhibition of the development, recurrence, onset, or spread of a disease, disorder, or condition, or a symptom thereof in a subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIG.s, and from the claims.

### DESCRIPTION OF DRAWINGS

FIGS. 1A-1B. Nb54 and Nb61 are selective for ALS-linked SOD1 variants over SOD1 WT in vitro. (FIG. 1A) Amino acid sequence alignment of Nb54 and Nb61. Sequence differences are highlighted in yellow. (FIG. 1B) Enzyme linked immunosorbent assays (ELISAs) were performed by coating the indicated SOD1 protein onto the well and increasing the concentration of Nb54 (left) or Nb61 (right) as described in the methods. The optical density (OD) at 450 nm correlates with Nb reactivity for the indicated SOD1 variant. Bars depict mean±standard deviation for three independent experiments. For each nanobody concentration (mg/ml), the OD at 450 nm for SOD1 A4V, G93A or Ox was compared to the corresponding value for SOD1 WT using a two-way ANOVA followed by Dunnett's multiple comparison test. **p<0.01, ***p<0.001, and ****p<0.0001.

FIGS. 2A-2I. Exogenous SOD1 levels are elevated upon co-expression with anti-SOD1 nanobodies. (FIGS. 2A-2C) Immunofluorescence images of HEK293T cells co-transfected with myc-tagged SOD1 WT (FIG. 2A), SOD1 A4V (FIG. 2B) or SOD1 G93A (FIG. 2C) and nanobodies (Nb54, Nb54-PEST, Nb61 or Nb61-PEST). An “empty” nanobody vector condition serves as a control for baseline SOD1-myc expression. Scale bar, 10 μm. (FIGS. 2D-2I) Quantification of SOD1-myc fluorescence signal intensity in cells co-transfected with nanobody and SOD1. (FIG. 2D) Co-transfection of Nb54 and Nb54-PEST leads to significantly enhanced SOD1 WT signal. (FIG. 2E) The same as in (FIG. 2D) except with Nb61 constructs. (FIG. 2F) Co-transfection of Nb54 and Nb54-PEST leads to significantly enhanced SOD1 A4V signal. (FIG. 2G) Neither Nb61 or Nb61-PEST had an effect on SOD1-myc signal intensity when co-transfected with SOD1 A4V. (FIG. 2H) Co-transfection of Nb54 and Nb54-PEST leads to significantly enhanced SOD1 G93A signal. (FIG. 2I) Same as (FIG. 2H) except with Nb61 constructs. For FIGS. 2D-2I, data is pooled from three biological replicates and represented with box and whisker plots, with boxes indicating the 25th (above) to 75th (below) percentiles and the median (line); whiskers denote the maximum and minimum values, respectively. Each point represents one cell (n refers to cell number beneath the plots); a different symbol is used for each biological replicate. Statistical analyses were performed with the Kruskal-Wallis test followed by Dunn's to correct for multiple comparisons; *p<0.05, **p<0.01, ***p<0.001, and ****p<0.0001. All significant comparisons are shown.

FIGS. 3A-3C. Exogenous expression levels of SOD1-myc and nanobody proteins are positively correlated in cellulo. Linear regression analyses of SOD1 and nanobody signal intensity. For all conditions there is a moderate positive correlation, with SOD1 and Nb54-PEST co-transfections demonstrating the strongest correlations. (FIG. 3A) HEK293T cells co-transfected with SOD1 WT and Nb54, Nb54-PEST, Nb61 or Nb61-PEST. (FIG. 3B) HEK293T cells co-transfected with SOD1 A4V and Nb54, Nb54-PEST, Nb61 or Nb61-PEST. (FIG. 3C) HEK293T cells co-transfected with SOD1 G93A and Nb54, Nb54-PEST, Nb61 or Nb61-PEST. For FIGS. 3A-3C: Data is pooled from three biological replicates with the exception of SOD1 WT co-transfected with Nb61 and Nb61-PEST, which are pooled from six biological replicates. Each point represents one cell, each symbol denotes a specific biological replicate. The r² values reflect the fit of experimental data to the depicted regression line. Increasing r² values indicate that the SOD1 and Nb signal intensities are positively correlated.

FIGS. 4A-4H. Nb54 and Nb61 restore the nucleocytoplasmic ratio of mutant SOD1 to SOD1 WT levels. (FIG. 4A) Immunofluorescence images of SOD1-myc signal in HEK293T cells co-transfected with SOD1 WT, SOD1 A4V or SOD1 G93A and nanobodies (Nb54 or Nb61) or an empty vector control. Scale bar, 10 μm. (FIG. 4B) Quantification of SOD1-myc N/C ratio in cells co-transfected with SOD1 WT, SOD1 A4V or SOD1 G93A and an empty vector control. SOD1 A4V and G93A have significantly reduced N/C ratios compared to SOD1 WT, indicating more cytoplasmic SOD1-myc signal. (FIGS. 4C-4E) Quantification of SOD1-myc N/C ratio in cells co-transfected with Nb54 or Nb54-PEST and SOD1 WT (FIG. 4C), SOD1 A4V (FIG. 4D) or SOD1 G93A (FIG. 4E). Co-transfection with Nb54 or Nb54-PEST increased the N/C ratios of both SOD1 variants, resulting in ratios similar to SOD1 WT, with the exception of Nb54-PEST with SOD1 G93A (E), (FIGS. 4F-4H) Quantification of SOD1-myc N/C ratio in cells co-transfected with Nb61 or Nb61-PEST and SOD1 WT (FIG. 4F) SOD1 A4V (FIG. 4G) or SOD1 G93A (FIG. 4H). Both Nb54 and Nb54-PEST significantly increased SOD1 A4V and SOD1 G93A N/C ratios to ratio values similar to SOD1 WT. For FIGS. 4B-4H, data is pooled from three biological replicates and represented with box and whisker plots, with boxes indicating the 25th (above) to 75th (below) percentiles and the median (line); whiskers denote the maximum and minimum values, respectively. Each point represents one cell (n refers to cell number beneath the plots); a different symbol is used for each biological replicate. Statistical analyses were performed with the Kruskal-Wallis test followed by Dunn's to correct for multiple comparisons; **p<0.01, ***p<0.001, and ****p<0.0001. All significant comparisons are shown.

FIGS. SA-5B. Binding of Nb54 and Nb61 confer stabilization to SOD1 A4V. Thermal denaturation profiles obtained by differential scanning fluorimetry (DSF) for SOD1 A4V and anti-SOD1 nanobodies alone and in complex. (FIG. 5A) SOD1 A4V (black curve), Nb54 (grey curve) and a mixture of SOD1 A4V and Nb54 (magenta curve). Addition of Nb54 to SOD1 A4V results in a higher temperature (Tₘ⁵) melting transition. Tₘ¹=43.8° C., Tₘ²=48.9° C., Tₘ³=60.0° C., Tₘ⁴=45.0° C., Tₘ⁵=71.1° C. (FIG. 5B) As in (A) except with Nb61 (grey curve). Melting profile of Nb61/SOD1 A4V complex shows a peak at a higher temperature (˜85° C.) compared to either SOD1 A4V or Nb61. Tₘ¹=48.6° C., Tₘ²=48.9° C., Tₘ³=60.0° C., Tₘ⁴=49.5° C. Data presented here are representative of n=3 separate experiments.

FIGS. 6A-6F. Nb61-PEST promotes neurite outgrowth in human SOD1 A4V motor neurons. SOD1 A4V iPSC-derived human motor neurons were thawed, plated into 384-well dishes and assessed under various conditions after 7 days in culture. (FIG. 6A) Immunofluorescence images of human SOD1 A4V neurons that were untreated (left) or treated (right) with a control lentivirus expressing GFP. Neurofilament heavy (NFH, SMI32) staining identified motor neurons. Scale bar=200 μm. (FIG. 6B) Quantification of the transduction efficiency for a control lentivirus expressing GFP or lentiviral constructs expressing either Nb61 or Nb61-PEST with a GFP reporter. Data are compiled from multiple wells (each point represents one well) from a representative biological replicate. (FIG. 6C) Immunofluorescence images of SOD1 A4V neurons that were transduced with the indicated lentivirus and stained as in (FIG. 6A). Scale bar=200 μm. (FIG. 6D) Quantification of total neurite length for SOD1 A4V neurons transduced with the indicated lentivirus revealed significantly enhanced neurite outgrowth upon expression of Nb61-PEST compared to the GFP control virus. Data are compiled from n=3 biological replicates; each replicate is denoted by a distinct symbol. (FIG. 6E) As in (FIG. 6C) with additional anti-SOD1 staining. Scale bar=25 μm. Arrows indicate cells that are GFP+, NFH+ and have clear SOD1 signal. (FIG. 6F) Quantification of endogenous SOD1 fluorescence signal intensity from images shown in (FIG. 6E) demonstrate that transduced Nb61 and Nb61-PEST lead to enhanced SOD1 expression in SOD1 A4V neurons. Data are pooled across n=3 biological replicates, with each point representing data acquired within a single well. For FIG. 6D and FIG. 6F, statistical analyses were performed with the Kruskal-Wallis test and Dunn's multiple comparison test; **p<0.01, ***p<0.001.

FIGS. 7A-7D. Nb61 constructs are nonlethal when expressed in human SOD1 WT motor neurons. SOD1 WT iPSC-derived human motor neurons were thawed, plated into 384-well dishes and assessed as described for SOD1 A4V neurons in FIG. 6. (FIG. 7A) Immunofluorescence images of SOD1 WT neurons, either non-transduced (left) or transduced (right) with a control lentivirus expressing GFP. Motor neurons were identified with anti-NFH staining. Scale bar=200 μm. (FIG. 7B) Quantification of the transduction efficiency for the indicated lentivirus from a representative biological replicate. (FIG. 7C) Immunofluorescence images of SOD1 WT iPSC-derived motor neuron cultures at DIV7 that were transduced with a control GFP-expressing lentivirus or lentivirus co-expressing Nb61 or Nb61-PEST and GFP. NFH staining was used to identify motor neurons. Scale bar=200 μm. (FIG. 7D) Quantification of total neurite length of SOD1 WT (n=2) iPSC-derived motor neurons that were transduced with a control lentivirus expressing GFP or lentivirus co-expressing Nb61 or Nb61-PEST and GFP. Neither Nb61 nor Nb61-PEST altered neurite length compared to SOD1 WT neurons transduced with control GFP lentivirus. Data represent three experimental replicates, each denoted by a different symbol. Kruskal-Wallis with Dunn's multiple comparison test was used for analysis; no significant comparisons were found.

FIGS. 8A-8B: Nb54 detects SOD1 from spinal cord lysate of transgenic SOD1 G93A mouse: (FIG. 8A) A schematic representation of the competitive ELISA that was used to assess the competition of various antigens with SOD1 G93A for binding to Nb54. An antigen that is reactive to Nb54 is expected to reduce binding of Nb54 to SOD1 G93A, which is immobilized on the plate. (FIG. 8B) Spinal cord lysate from SOD1^{G93A} transgenic mice (green line) expressing both human SOD1 G93A and endogenous murine SOD1 WT competes with (i.e., reduces) Nb54 binding to immobilized SOD1 G93A in a dose-dependent manner, whereas lysate from non-transgenic (Non-Tg; dark grey) mice expressing only endogenous murine SOD1 WT does not. Dilutions of Non-Tg lysate (light purple) or buffer spiked with recombinant SOD1 G93A (0.2 μg/25 μl; dark purple) were used as positive controls for competition of Nb54 binding to immobilized SOD1 G93A. Error bars depict standard deviation among n=4 animals per genotype. A two-way ANOVA was performed for all lysate samples followed by a Dunnett's multiple comparison test to compare SOD1^{G93A} transgenic lysate or Non-Tg spiked with SOD1 G93A to the Non-Tg mouse lysate. **p<0.01, ***p<0.001, and ****p<0.0001.

### DETAILED DESCRIPTION

Described herein are nanobodies derived from llama sera that exhibit selective reactivity for misfolded SOD1 proteins compared to SOD1 WT. Anti-SOD1 nanobodies did not reduce expression levels of misfolded SOD1 protein in mammalian cells, but rather appear to stabilize the misfolded conformation of mutant SOD1 in cells and in vitro. Co-expression of anti-SOD1 nanobodies lead to increased levels of mutant SOD1 in mammalian cells, as well as enhanced nuclear-to-cytoplasmic (N/C) localization of mutant SOD1 to levels that are similar to SOD1 WT. Importantly, expression of anti-SOD1 nanobodies exerted a beneficial effect on the health of neurons derived from ALS-human induced pluripotent stem cells (iPSCs). These data demonstrate that anti-SOD1 nanobodies have therapeutic potential for modifying the pathogenic properties of mutant SOD1 proteins in vivo.

### SOD1:

Protein accumulation, modifications and aggregation are pathological aspects of numerous neurodegenerative diseases such as Huntington's, Alzheimer's (AD) and Parkinson's diseases (PD). Misfolding, aggregation and precipitation of proteins seem to be directly related to neurotoxicity in these diseases. The native homodimeric, copper-zinc superoxide dismutase (SOD1) protein (both wild-type and mutants that result in ALS) has a tendency to form fibrillar aggregates in the absence of the intramolecular disulfide bond or of bound zinc ions. Related to misfolded/aggregated SOD1 are disorders such as amyotrophic lateral sclerosis (ALS), also known as Lou Gehrig's disease, or Charcot's disease. Further, oxidative modifications of SOD1 which may also induce the protein's misfolding have been found in AD and PD, and aggregates of SOD1 are associated with amyloid plaques and neurofibrillary tangles in AD patients implicating a possible role of SOD1 in the pathology of these diseases.

The term “SOD1” as used herein refers to the gene superoxide dismutase 1 and its encoded protein (Gene ID: 6647 for the human gene). The enzyme SOD1 binds copper and zinc ions and is one of three superoxide dismutases responsible for destroying free superoxide radicals in the body. Mutations in this gene have been linked to familial amyotrophic lateral sclerosis, and several pieces of evidence also show that wild-type SOD1, under conditions of cellular stress, is implicated in a significant fraction of sporadic ALS cases. Over 170 mutations of SOD1 have been linked to ALS; “mutant SOD1,” in particular, refers to SOD1 containing one or more mutations that are linked to ALS. Selected examples (listed as one-letter amino acid abbreviations, with numbering referring to the human protein) include those listed in the OMIM database under entry 147450, i.e., A4V, G93A, H46R, H48Q, G85R, D90A, and I113T.

As recited here, the term “SOD1”, is used interchangeably to specifically refer to the native monomer or dimeric form of SOD1. The term “SOD1” is also used to generally identify other conformers of SOD1, for example, oligomers or aggregates of SOD1. The term “SOD1” may also be used to refer collectively to all types and forms of SOD1.

An exemplary protein sequence for human SOD1 is:

(SEQ ID NO: 1)
MATKAVCVLKGDGPVQGIINFEQKESNGPVKVWGSIKGLTEGLHGFHVH
EFGDNTAGCTSAGPHFNPLSRKHGGPKDEERHVGDLGNVTADKDGVADV
SIEDSVISLSGDHCIIGRTLVVHEKADDLGKGGNEESTKTGNAGSRLAC
GVIGIAQ.

The amino acid sequence of SOD1 of 154 aa can be retrieved from the literature and pertinent databases; see, e.g., Sherman et al., Proc. Natl. Acad. Sci. USA. 80 (1983), 5465-9; Kajihara et al., J. Biochem. 104 (1988), 851-4; GenBank SOD1 Homo sapiens, accession number CAG46542. The “wild type” or recombinant human SOD1 amino acid sequence is represented by the above mentioned sequence according to SEQ ID NO:1.

### Single-Domain Antibodies:

Naturally occurring human antibodies are heterotetramers. The antibodies provided herein in one aspect comprise an antigen binding site in a single polypeptide. The antibodies are therefore herein referred to as “single domain antibodies”. Single domain antibodies are also known as nanobodies. The single antibodies disclosed herein may, though, in certain embodiment be bispecific or multispecific single domain antibodies as described elsewhere herein, where two single domain antibodies are coupled.

A single domain antibody is an antibody fragment consisting of a single monomeric variable antibody domain. Like a whole antibody, it is able to bind selectively to a specific antigen. Single domain antibodies typically have molecular weights in the range of 12-15 kDa, i.e. much lower than common antibodies, ranging typically from 150 to 160 kDa. Single domain antibodies are also smaller than Fab fragments (˜50 kDa) of heterotetrameric antibodies comprising one light chain and half a heavy chain.

Single domain antibodies can be derived from antibodies found in nature, for example in camelids (VHH) and cartilaginous fishes (VNAR). New or Nurse Shark Antigen Receptor (NAR) protein exists as a dimer of two heavy chains with no associated light chains. Each chain is composed of one variable (V) and five constant domains. The NAR proteins thus constitute a single immunoglobulin variable-like domain. Single heavy-chain antibodies are also found in camelids, such as such as dromedaries, camels, llamas and alpacas, where the heavy chain has lost one of its constant domains and underwent modifications in the variable domain, both of which are structural elements necessary for the binding of light chains.

However, single domain antibodies can also be engineered by recombinant methods. One approach is to split the dimeric variable domains from common immunoglobulin G (IgG) from humans or mice into monomers. Single domains, which are derived from light chains, also bind specifically to target epitopes. Thus, the single domain antibody may be derived from any suitable organism.

Single domain camelid antibodies are equal to regular antibodies in terms of specificity. Single domain antibodies are easily isolated, for example by using phage panning procedures. The smaller size and single domain architecture make these antibodies easier to express as proteins in bacterial cells for large scale production, making them ideal for commercial exploitation. The antibodies of the present invention are therefore single domain antibodies, preferably derived from camelid antibodies, preferably llama antibodies, including functional homologs, fragments thereof and fusion macromolecules containing VHH covalently linked to glycan, nucleic acid, protein, or chemical groups not being a macromolecule.

The single domain VHH antibodies of the present invention preferably comprise one or more CDRs. In particular, the CDRs may identify the specificity of the antibody and accordingly it is preferred that the antigen binding site comprises one or more CDRs, preferably at least 1, more preferably at least 2, yet more preferably 3 or more CDRs. In one embodiment, the single domain antibody comprises 1 CDR. In one embodiment, the single domain antibody comprises 2 CDRs. In one embodiment, the single domain antibody comprises 3 CDRs.

mis-SOD1 Nanobodies

Provided are single-domain antibodies (or nanobodies) against misfolded SOD1, i.e., they recognize an epitope that is present in a mutated form of the SOD1 protein. According to particular embodiments, SOD1 is human SOD1. According to further particular embodiments, the mutant SOD1 is characterized by a mutation of amino acids at positions 4 and/or 93, particularly by an A4V and/or G93A mutation. According to yet further embodiments, the single-domain antibodies bind both wild-type and mutant SOD1 (i.e., they recognize an epitope present in the wild-type protein and at least two (but possibly more) mutated isoform). In the case of SOD1 A4V and G93A, the single-domain antibody has a higher selectivity and/or affinity to the mutant SOD1 than wild-type SOD1 in vitro.

According to particular embodiments, the single-domain antibody is an inhibitory single-domain antibody against SOD1. Typically, this means that the nanobody interferes with the superoxide dismutase function of SOD1. However, according to particular embodiments, the inhibitory single-domain antibody inhibits the toxic gain of function activity resultant from the mutant SOD1 protein. Most particularly, the single-domain antibody interferes with (inhibits, prevents, reverses or slows) the formation of SOD1 aggregates; and/or the single-domain antibody can counter the phenotypic changes caused by expression of the mutant SOD1 protein (e.g., axonopathy).

According to particular embodiments, the single-domain antibody has a sequence selected from the group of SEQ ID NOS: 2-24.

A33025
(SEQ ID NO: 2)
QVQLVESGGGWVQTGGSLKLSCVVSGINFADSRMGWYRQAPGNQYDPIAEMNVGGLRKYADS
VKTRFTISRNNVKNTVYLQMDSLKPEDTGVYVCGAETIWDSARYWGQGIQVTVSS
A33151
(SEQ ID NO: 3)
HVQLVESGGGSVQAGGSLRLSCVVSGINFGDSAAGWYRQVPGQLREFVASLSRSGERNFADS
VKDRESMSRVNAKNTVFLQMNDLKVEDTAVYYCNVGDTLPSQYWGQGTQVTVSS
A35386
(SEQ ID NO: 4)
AVQLVDSGGGSVQAGGSLRLSCVVSGIDFGDSAAGWYRQVPGQLREFVASLSRSGERNFADS
VKDRFSMSRVNAKNTVFLQMNDLKVEDTAVYYCNVGDTLPSQYWGQGTQVTVSS
A35353
(SEQ ID NO: 5)
EVQLVESGGGSVQAGGSLRLSCVVSGINFADSRMGWYRQAPGQLREFVASLSRSGERNFADS
VKDRESMSRVNAKNTVFLQMNDLKVEDTAVYYCNVGDTLPSQYWGQGTQVTVSS
A33153
(SEQ ID NO: 6)
QVQLVESGGGWVQAGGSLRLSCVVSGTNFNDRSMGWYRQAPGKQRELVATMSFGGRRNYVDA
VKARFTISRDNRKNTTYLLMNDLEPDDTAVYYCAAGHVYASVAPATTWIEYWGQGTQVTVSS
A33161
(SEQ ID NO: 7; also referred to herein as “Nb61”)
QVQLVESGGGWVQAGGSLRLFCVVSGTNFNDRSMGWYRQAPGKERELVATMSYGGRRNYADA
VKARFTISRDNRKNTTYLLMNDLEPDDTAVYYCAAGHVLESVVPATTWIEYWGQGTQVTVSS
A35349
(SEQ ID NO: 8)
QVQLVESGGGWVQAGGSLRLSCVVSGTNFNDRSMGWYRQAPGKERELVATLSYGGRRNYVDA
VKARFTMSRDNRKNTTYLLMNDLEPDDTAVYYCAAGHVFESVVPATTWIEYWGQGTQVTVSS
A35354
(SEQ ID NO: 9; also referred to herein as “Nb54”)
QVQLVESGGGWVQAGGSLRLSCVVSGTNFNDRSMGWYRQAPGKERELVATLSYGGRRNYVDA
VKARFTMSRDNRKNTTYLLMNDLEPDDTAVYYCAAGHVLESVVPATTWIEYWGQGTQVTVSS
A35388
(SEQ ID NO: 10)
QVQLVESGGGSVQAGGSLRLSCAHSGSNFNDRSMGWYRQAPGKERELVATLSYGGRRNYVDA
VKARFSMSRDNRKNTTYLLMNDLEPDDTAVYYCAAGHVLESVVPATTWIEYWGQGTQVTVSS
A33160
(SEQ ID NO: 11)
QVQLVESGGGLVQAGGSLRLSCAASGIPFNEKIMGWYRQAPGNERELVAYIDTTSRTWHKDY
LRDRFTISRDNAKNAAYLQMNSLKPEDTAVYYCATLWNTPTNYWGKGTQVTVSS
A35387
(SEQ ID NO: 12)
QVQLVESGGGLVQAGGSLRLSCAASGIPFNEKIMGWYRQAPGNERELVAYIDTTSRTWHKDY
LRDRFTISRDNAENAAYLQMNSLKPEDTAVYYCATLWNTPTNYWGKGTQVTVSS
A35367
(SEQ ID NO: 13)
QVKLEESGGGLVQAGESRRLSCAPSGRIFGINGMGWYRRTPGKQRETVAILYSDGTITYGDS
VKGRFTISRDNANGTPSLQMTDLKPEDAGLYICNVGGHWGSGTQVTVSS
A33149
(SEQ ID NO: 14)
QVQLVESGGGLVQAGGSLRLSCAASGSVNVMGWYRQAPGRERTIVAHITSGSTTTYVDSVKG
RFTISRDNAKNTVYLQMNSLKPEDTAVYYCAAEGAFTGWGPEYWGQGTQVTVSS
A35467
(SEQ ID NO: 15)
QVQLVESGGGLVQAGGSLRLSCAASGSVNVMGWYRQAPGRERTLVAHITSGSTTTYVDSVKG
RFTISRDNAKNTVYLQMNSLKPEDTAVYYCAAEGAFTGWGPEYWGQGTQVTVSS
A33203
(SEQ ID NO: 16)
QVKLEESGGGLVQAGGSLRLACTASGRIRNINAMGWYRQAPGRERTLVAHITSGSTTTYVDS
VKGRFTISRDNAKNTVYLQMNSLKPEDTAVYYCAAEGAFTGWGPEYWGQGTQVTVSS
A33198
(SEQ ID NO: 17)
QVQLVESGGGLVQPGGSLRLSCAASGSDAILNVMGWYRQAPGWERTLVGHITSGGTTTYVDA
VKGRFTISRDNAENTVYLQMNSLKPEDTAVYYCAAEGAFTGWGPDYWGQGTQVTVSS
A35470
(SEQ ID NO: 18)
QVQLVESGGGLVQPGGSLRLSCAASGSDAILNVMGWYRQAPGWERTLVGHITSGGTTTYVDA
VKGRFTISRDNAKNTVYLQMNSLKPEDTAAYYCAAEGAFTGWGPDYWGQGTQVTVSS
A35395
(SEQ ID NO: 19)
QVQLVESGGGLVEAGGSLRLSCAASGSDAILNVMGWYRQAPGWERTLVGHITSGGTTTYVDA
VKGRFTISRDNAKNTVYLQMNSLKPEDTAVYYCAAEGAFTGWGPDYWGQGTQVTVSS
A33152
(SEQ ID NO: 20)
QVQLVESGGGLVQAGGSLRLSCIASGSFLSINVMGWYRQAPGKQRELVGHITKGGTTTYTDS
VKGRFTISRDNAKNTVYLQMNSLKLEDTAVYYCAAEGAFTGWPPEYWGQGTQVTVSS
A35346
(SEQ ID NO: 21)
QVKLEESGGGLVQAGGSLRLSCIASGSFLSINVMGWYRQAPGKQRELVGHITKGVTTTYTDS
VKGRFTISRDNAKNTVYLQMNSLKLEDTAVYYCAAEGAFTGWPPEYWGQGTQVTVSS
A35494
(SEQ ID NO: 22)
QVQLVESGGGLVQAGGSLRLSCVASGSYPNVMGWYRQAPGKQRLLVAHITSGGTTTYADSVK
GRFTISRDNAKNTVYLQMNSLKLEDTAVYYCAAEGAFTGWGPEFWGQGTQVTVSS
A35509
(SEQ ID NO: 23)
QVQLVESGGGLVQPGGSLRLSCAASGSYPNVMGWYRQAPGKQRLLVAHITSGGTTTYADSVK
GRFTISRDNAQNTVYLQMNSLKLEDTAVYYCAAEGAFTGWGPEFWGQGTQVTVSS
A35513
(SEQ ID NO: 24)
QVQLVESGGGLVQAGESLRLSCAASGSNLDINVMGWYRQAPGKQRLLVAHITRGGSTTYADS
VKGRFTISRDNDKNTVYLQMNSLKPEDTAVYYCAAEGAFTGWGPEYWGQGTQVTVSS

The single-domain antibodies provided herein also include functional variants thereof. The term “functional variant” is meant to include those variants, which retain some or essentially all the ability of an antibody to selectively binding its antigen or ligand, such as any of the ligands mentioned herein below. Functional variants include any variant, that is at least 75% identical to a single-domain antibodies provided herein, such as at least about 75, 76, 77, 78, 79, 80, 81, 82, 83, 84, 85, 86, 87, 88, 89, 90, 91, 92, 93, 94, 95, 96, 97, 98, 99, 99.5% identical to a single-domain antibody provided herein, such as any of those identified by SEQ ID NOs: 2-24.

### Fusion Proteins (i.e., Labeled Mis-SOD1 Nanobodies for Methods of Monitoring Motor Neuron Pathologies)

In some embodiments, the mis-SOD1 nanobodies are fused to a tag or label. For instance, the tag to which the single-domain antibody is fused is a His-tag, HA-tag, Myc-tag, degradation tag, and/or a radiolabel tag. In some instances, N-terminal additions may be preferred and can be selected from any relevant additional moieties, depending on the contemplated application of the antibody and the desired functionalities to the final antibody product. For instance, Albumin may be added for increasing circulation time and protect the product from degradation. Other antigen binding fragments, single domain antibodies or fragments thereof may also be added for introducing a second affinity/binding specificity to the antibody product.

Further, SOD1 normally is a soluble cytoplasmic protein, although a detrimental or neurotoxic role has been ascribed both to extracellular secreted SOD1 and cytoplasmic mutant SOD1. In order to be able to inhibit intracellular forms of SOD1, according to particular embodiments, the mis-SOD1 nanobody is able to enter cells, particularly neuronal cells. This may be an inherent property of the nanobody, may be achieved by the addition of a suitable moiety or tag that allows cellular uptake, or may be directly injected into a cell (i.e., transfected).

In some embodiments, the mis-SOD1 antibodies are fused to a degradation tag. As used herein, the term “degradation tag” refers to an amino acid sequence that promotes degradation of an attached protein through either the proteasome or autophagy-lysosome pathways. In some embodiments, a degradation tag (also known as a degradation sequence or a degradation signal) is a polypeptide that destabilizes a protein such that half-life of the protein is reduced (e.g., reduced at least two-fold), when fused to the protein.

Many different degradation tags are known in the art. Any degradation tag known in the art can be fused to any of the mis-SOD1 antibodies described herein. Non-limiting examples of degradation tags include PEST sequences, HCV NS4 degrons, APC/C degrons (e.g., D box, KEN box and ABBA motif), KEAP1 binding degrons, MDM2 binding motifs, N-degrons (e.g., Nbox, or UBRbox), and phospho-dependent degrons.

In some embodiments, the mis-SOD1 antibodies are fused to a PEST sequence, which is an amino acid sequence that targets a protein to the ubiquitin-proteosome pathway for degradation. In some embodiments, the PEST sequence is flanked by clusters containing several positively charged amino acids. Non-limiting examples of PEST sequences that can be used as described herein include those provided in Rechsteiner and Rogers (1996) Trends Biochem. Sci. 21:267-271, which is incorporated by reference for the purposes and subject matter referenced herein.

In particular embodiments, the mis-SOD1 antibodies are fused to a radiolabel, such as ⁶⁴Cu, ⁶⁷Ga, ⁸⁶Y, ¹²⁴I, ¹²⁵I, ¹¹¹In, ⁸⁹Zr, or ^{99m}Tc.

Provided herein are non-invasive methods for diagnosing a motor neuron disorder in a living subject, e.g., a subject presenting with one or more symptoms of a neurodegenerative disorder or a subject not presenting a symptom of a neurodegenerative disorder (e.g., an undiagnosed and/or asymptomatic subject). More particularly, provided herein are methods of monitoring the progression of ALS (or ALS symptoms) in a subject. Also provided herein are prognostic methods and methods of monitoring progression of a motor neuron pathology (e.g., ALS), as well as methods of determining whether a treatment for a motor neuron pathology is having any therapeutic effect, e.g., decreasing the rate of onset or the progression of the disease.

Subjects associated with predetermined values are typically referred to as reference subjects. For example, in some embodiments, a control reference subject does not have a disorder that entails motor pathology, such as motor neuron pathology.

A disease reference subject is one who has (or has an increased risk of developing) pathology of the motor neurons, such as motor neuron pathology or neuropathy. An increased risk is defined as a risk above the risk of subjects in the general population.

The methods described herein can use any imaging modality suitable for imaging the labeled agents in living subjects. Suitable imaging methods include nuclear imaging method such as computed tomography (CT), magnetic resonance/nuclear magnetic resonance imaging (MRI/NMR), Single photon emission computed tomography (SPECT) or positron emission computed tomography (PET), using an agent labeled, e.g., with ⁶⁴Cu, ⁶⁷Ga, ⁸⁶Y, ¹²⁴I, ¹²⁵I, ¹¹¹In, ⁸⁹Zr, or ^{99m}Tc. See, e.g., Den et al., Nucl Med Biol. 2013 January; 40(1): 3-14.

The labeled mis-SOD1 nanobodies can be produced using methods known in the art, e.g., using standard protein production (e.g., by recombinant expression in vitro) and purification methods, and labeled using known chemistries, e.g., as described herein or known in the art.

### Nucleic Acids, Host Cells, Kits

According to particular embodiments, the single-domain antibodies are not provided as such, but are provided as nucleic acid molecules, i.e., nucleic acid molecules encoding single-domain antibodies against mutant SOD1 as herein described. Also provided are vectors comprising such nucleic acids or nucleic acid molecules. According to yet further embodiments, host cells are provided comprising such nucleic acids or such vectors.

According to further embodiments, kits are provided comprising a single-domain antibody against mutant SOD1 and a pharmaceutically acceptable excipient. According to particular embodiments, the single-domain antibody (or fusion proteins comprising the single-domain antibody) may be provided as protein, as a nucleic acid encoding a single-domain antibody against mutant SOD1, or as a vector comprising such nucleic acid.

### Pharmaceutical Compositions

According to a further aspect, the single-domain antibodies are provided herein for use in medicine. In other words, the single-domain antibodies against mutant SOD1 are provided for use as a medicament. The same goes for the nucleic acid molecules encoding the single-domain antibodies, or for the vectors containing such nucleic acids. According to particular embodiments, the single-domain antibodies (or nucleic acids encoding them, or vectors comprising such nucleic acids) are provided for use in treatment of amyotrophic lateral sclerosis (ALS).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration, the present methods will typically include local intramuscular injection thus formulation for parenteral administration is desirable.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The pharmaceutical compositions can be included in a container, pack, or dispenser together with instructions for administration.

Also provided herein are methods for treating ALS, or of improving symptoms of ALS, in a subject in need thereof, comprising administering a single-domain antibody (or fusion protein comprising the single-domain antibody) against mutant SOD1 to the subject. Here also, the single-domain antibody may be provided as protein, or may be administered as a nucleic acid molecule encoding a single-domain antibody against mutant SOD1, or as a vector comprising such nucleic acid molecule. If the single-domain antibody is administered as protein, it is particularly envisaged that it is administered intracerebroventricularly, such as, e.g., through injection or pump. In some instances, methods additionally comprise administering single-domain antibody (or fusion proteins comprising the single-domain antibody) with a neurotrophin.

Neurotrophins or neurotrophic factors are a family of proteins that can induce the survival, development and function of neurons (e.g., sensory and sympathetic neurons) in both the peripheral and central nervous systems. Neurotrophins can activate one or more of the three members of the tropomyosin-related kinase (Trk) family of receptor tyrosine kinases (TrkA, TrkB, and TrkC). In addition, neurotrophins activate p75 neurotrophin receptor (p75NTR), a member of the tumor necrosis factor receptor superfamily. Through Trk receptors, neurotrophins activate Ras, phosphatidyl inositol-3 (PI3)-kinase, phospholipase C-gamma1 and signaling pathways controlled through these proteins, such as the MAP kinases. Activation of p75NTR results in activation of the nuclear factor-kappaB (NF-kappaB) and Jun kinase as well as other signaling pathways. Continued presence of the neurotrophins is required in the adult nervous system, where they control synaptic function and plasticity, and sustain neuronal survival, morphology and differentiation.

Neurotrophins suitable for linking to the clathrin nanoparticles described herein include but are not limited to: nerve growth factor (NGF), brain-derived neurotrophic factor (BDNF), neurotrophin-3 (NT-3) and neurotrophin-4 (NT-4), NT-6, NTN, PSPN, ARTN, CNTF, and LIF. Additional proteins that regulate neuronal survival and/or other aspects of neuronal development are also contemplated herein, such as but are not limited to glial cell-derived neurotrophic factor.

In some instances, the neurotrophin linked to clathrin nanoparticles described herein is BDNF. BDNF proteins can be unstable and do not easily cross the BBB (See. e.g., Gilmore et al. J Neuroimmune Pharmacol 3 (2): p. 83-94, 2008). BDNF has a short in vivo half-life (<5 min) and poor pharmacokinetic profile, which makes treatment with BDNF difficult. However, agents such as antidepressants and mood stabilizers that can increase BDNF levels act on different sites and have multiple side effects (See, e.g., Bhaskar et al. Part Fibre Toxicol. 7: p. 3, 2010). Without wishing to be bound by theory, linking BDNF to clathrin nanoparticles as described herein allows BDNF to be delivered across the BBB.

In case the single-domain antibody (or fusion proteins comprising the single-domain antibody) is provided as a nucleic acid or a vector, it is particularly envisaged that the single-domain antibody is administered through gene therapy.

### EXAMPLES

The invention is further described in the following examples, which do not limit the scope of the invention described in the claims.

### Materials and Methods for Examples 1-6

Generation of anti-SOD1 nanobodies: All recombinant SOD1 proteins used throughout this study, including antigens for the generation of anti-SOD1 nanobodies, were expressed and purified as described previously by our lab [6,38]. SODox was generated as described [38]. To create anti-SOD1 nanobodies, two llamas were immunized with SOD1 WT, familial-ALS linked SOD1 G93A and SOD1ox by Triple J Farms/Kent Laboratories (Bellingham, WA). Blood samples collected from both immunized animals were used to construct a nanobody gene library by GenScript USA Inc. through a single domain antibody (sdAb) library construction and binder discovery package SC1590. Briefly, total RNA was extracted from llama blood samples using Trizol. Nanobody encoding genes (V_{H}H) were RT-PCR cloned and amplified from the mRNA of peripheral blood mononuclear cells (including B-cells). The library was constructed by transformation of nanobody/V_{H}H DNA fragments into phage display SS320 chemically competent E. coli cells. Based on the number of transformants on the agar plates, the library size was estimated at >1.75×10⁹ unique sequences/clones. The library was then screened using phage display by the CRO GenScript USA Inc. for clones that exhibit selectivity for SODox and counter-screened to exclude clones that exhibit high reactivity for the native SOD1 WT protein. Two clones that exhibited >5-fold selectivity for SODox over the native SOD1 WT by enzyme-linked immunosorbent assay (ELISA) were pursued for additional studies.

Plasmid construction: Nanobody sequences were sub-cloned into the pTP212 plasmid (kind gift from Dr. Dirk Gorlich, Max Planck Institute for Biophysical Chemistry, Germany) for recombinant bacterial expression of nanobody protein containing an N-terminal His-SpbrNEDD8 tag [64]. The plasmid encoding the His-MBP-brSUMO-bdNEDP1 enzyme, which cleaves the SpbrNEDD8 tag, was also a kind gift from Dr. Dirk Gorlich [65]. Nanobody plasmids with and without a C-terminal PEST signal sequence (SHGFPPEVEEQDDGTLPMSCAQESGMDRHPAACASA RINV) for transient transfection into mammalian cells were synthesized as DNA G-blocks and then sub-cloned into the pcDNA 3.1 (−) plasmid (ThermoFisher Scientific, V87520) using the NEBuilder HiFi assembly kit (NewEngland Biolabs, E5520S). The same nanobody sequences (with and without PEST) were sub-cloned into the low expression lentivirus vector CShPW2 for lentiviral expression using the NEBuilder HiFi assembly kit (NewEngland Biolabs, E5520S). The CShPW2 plasmid contains a green fluorescent protein (GFP) reporter expressed downstream of an internal ribosome entry site (IRES) and independently of the nanobody sequence [66]. Plasmids for mammalian expression of SOD1-myc under the cytomegalovirus (CMV) promotor were a kind gift from Dr. Zuoshang Xu (University of Massachusetts Chan medical school, USA).

Recombinant nanobody expression and purification: With the exception of the competition ELISA, all in vitro experiments were performed with nanobody protein prepared as follows. Nanobodies with N-terminal His-SpbrNEDD8 tag were expressed in Escherichia coli (E. coli) BL21 (DE3) pLysS cells (Millipore Sigma, 69451-3). Bacterial cultures (IL) were grown at 37° C. until the optical density (OD) reached 0.6-0.7. Protein expression was induced by adding isopropyl-beta-D-thiogalactoside (IPTG, Goldbio, I2481C25) to a final concentration of 1 mM. The cultures were further grown at 16° C. for 16h, after which the cells were harvested by centrifugation and stored at −80° C. until the purification could be initiated.

Bacterial pellets were thawed on ice and resuspended in chilled lysis buffer (50 mM Tris/HCl, 500 mM NaCl, 10 mM imidazole, 1 mg/ml lysozyme, 0.3% NP-40, pH 7.4) supplemented with protease inhibitor cocktail (Millipore Sigma, 11873580001). After sonication, the lysates were clarified by centrifugation at 26,000×g for 30 min at 4° C. The clarified lysate was loaded onto a 1 mL HisTrap HP column (Cytiva, 29051021) equilibrated with buffer A (50 mM sodium phosphate, 300 mM NaCl, 45 mM imidazole, pH 7.0) and subsequently washed with buffer A. Bound proteins were eluted with 50 mM sodium phosphate, 300 mM NaCl and 500 mM imidazole at pH 7.0. Elution fractions containing nanobody were pooled and concentrated using a centrifugal concentrator (Vivaspin 5000 MWCO, Sartorius, VS0611) as per the manufacturer's instructions. The concentrated protein was buffer exchanged into phosphate buffered saline (PBS, pH 7.4) using a Sephadex-25 desalting column (Cytiva, 17085101). To cleave the His-SpbrNEDD8 tag from the nanobodies, His-MBP-brSUMO-brNEDP1 enzyme was expressed and purified similar to the above protocol. Tag cleavage was performed by incubating nanobody proteins with His-MBP-brSUMO-brNEDP1 enzyme at molar ratio of 1:100 (enzyme: nanobody) in PBS containing 0.25M sucrose, 2 mM MgCl₂ and 2 mM dithiothreitol (DTT) for 90 min at 4° C. The mixture was applied to the 1 mL HisTrap HP re-equilibrated with buffer A and the flow through containing the untagged nanobody was collected. Untagged nanobody was concentrated and buffer exchanged into PBS as described above and stored at −80° C. For the competitive ELISA, nanobody with a non-cleavable his-tag was purified similarly to the protocol above.

ELISA: An indirect ELISA was used to assess the selectivity of the nanobodies for recombinant SOD1 proteins as follows. SOD1 (0.1 μg/50 μl in phosphate buffered saline; PBS) was coated onto 96 well medium binding microplates (Greiner BioOne, 655001) overnight at 4° C. All subsequent steps were performed at ambient temperature. Coated plates were washed with wash buffer (PBS containing 0.05% (v/v) Tween-20) and blocked with a 5% (w/v) solution of bovine serum albumin (BSA) in PBS for 1 h. After washing, the plates were incubated for 1.5h with nanobody diluted (0-1 μg/ml) in wash buffer. Plates were then washed three times with wash buffer and incubated with horseradish peroxide conjugated anti-nanobody (1:2500, GenScript, U8401BI120) for 1 h. After three washes, 100 μl of 3,3′,5,5′-Tetramethylbenzidine (TMB, SurModics, TMBS-0100-01) was added for 10-30 min. The reaction was quenched with 100 μl Liquid Stop solution (Surfmodics, LSTP-0100-01). The absorbance or optical density (OD) at 450 nm for each well was measured with a plate reader (Perkin Elmer, Victor X5 model). The average of the OD 450 nm for each independent ELISA experiment (n=3) was determined and statistical analysis was performed as described below and in the figure legend.

A competitive ELISA was used to assess a potential interaction between nanobody and SOD1 derived from an ALS mouse model as follows. Spinal cord tissue was extracted from four P70-78 B6SJL-Tg (SOD1*G93A)1Gur/J (SOD1^{G93A}) mice and four P70-75 WT [4], non-transgenic mice. Tissues were lysed separately in ice cold 25 mM Tris/Cl, pH 7.8 buffer supplemented with protease inhibitor cocktail (Millipore Sigma, 11873580001) using a Dounce homogenizer. Lysates were clarified by centrifugation at 15,600×g, 4° C. and the total protein concentration was determined by the bicinchoninic acid assay (BCA; Thermofisher, 23227) according to the manufacturer's instructions. All research involving animals for the following post-mortem tissue processing was reviewed and approved by the Institutional Animal Care and Use Committee (IACUC) at the University of Massachusetts Chan Medical School.

Microplates (96-well) were coated with recombinant SOD1 G93A (0.1 μg/50 μl diluted in PBS) and blocked with BSA as described above. Murine tissue lysates (25 μl of 3.6 mg/ml) either alone or spiked with recombinant SOD1 G93A (0.2 μg/25 μl) were added to the coated wells. Serial dilutions were prepared in 25 μl of assay buffer (0.2% (w/v) BSA in PBS). Recombinant SOD1 G93A (0.2 μg/25 μl) diluted in assay buffer served as a positive control for competition. 25 μl of Nb54 (0.2 μg/ml) diluted in assay buffer was added to all the wells and incubated at ambient temperature for 1h. Plates were washed and processed as described for the indirect ELISA, except absorbance values were normalized to the signal from Nb54 applied to wells coated with SOD1 G93A in the absence of competing antigen. A statistical analysis was performed as described below and in the figure legend.

Transient transfection of HEK293T cells: Human embryonic kidney 293T (HEK293T) cells were maintained in Dulbecco's modified Eagle's medium (DMEM; Invitrogen, 11965118) containing 10% (v/v) fetal bovine serum (MilliporeSigma, catalog no. F4135) and 1% (w/v) penicillin-streptomycin (Invitrogen, 15140122) at 37° C. and 5% CO₂. Cells at a density of 1.6×10⁵ cells/well were plated in a 24-well plate containing coverslips coated with poly-l-lysine. 24h later, cells were transiently co-transfected with plasmids encoding nanobody or nanobody-PEST (1 μg) and SOD1-myc, SOD1-A4V-myc or SOD1-G93A-myc (50 ng) using 3 μl Lipofectamine 2000 (ThermoFisher, 11668-019) diluted in OptiMEM (Invitrogen, 31985070). Control conditions were included as follows: cells transfected with Nb54 or Nb54-PEST alone; cells transfected with Nb61 or Nb61-PEST alone; cells co-transfected with an empty vector (the nanobody plasmid without the nanobody gene) and either SOD1-myc, SOD1 A4V-myc or SOD1-G93A-myc.

Cells were fixed 24h post-transfection with 4% paraformaldehyde for 15 min at ambient temperature. For immunofluorescence microscopy, cells were permeabilized with 1% Triton X-100 (Sigma, T9284) for 10 minutes and blocked with PBSAT (PBS with 1% BSA and 0.5% Triton X-100) for 1h. Cells were incubated with the rabbit anti-nanobody (1:1000) described above and mouse anti-myc (1:100) for 1h 15 min. Anti-myc (9E 10) was developed by Bishop, J. M., University of California, San Francisco (UCSF), and was obtained from the Developmental Studies Hybridoma Bank (DSHB), created by the National Institute of Child Health and Human Development (NICHD) of the National Institutes of Health (NIH) and maintained at The University of Iowa (Iowa City, IA), Department of Biology. Cells were then incubated with secondary antibodies anti-rabbit Alexa Fluor 488 (Jackson ImmunoResearch Laboratories, 711-545-152) and anti-mouse Cy3 (Jackson ImmunoResearch Laboratories, 715-165-151) diluted 1:2000 in PBSAT for 1 h. To outline and define the cell boundary, cells were stained with Phalloidin Alexa Fluor 647 (Invitrogen, A22287) at 1:100 in PBSAT for 40 min, and then counterstained with DAPI (Sigma Aldrich, D9542) for 5 min. Coverslips were mounted using Prolong Gold anti-fade reagent (Cell Signaling Technologies, 9071S) with a refractive index of 1.46.

Image acquisition and analysis of transfected HEK293T cells: Images were acquired with a Leica DMI 6000 inverted fluorescent microscope equipped with a 40× air lens and a Leica DFC365 FX camera (6.45-μm pixel size) using AF6000 Leica software v.3.1.0 (Leica Microsystems). Twelve μm z-stacks (0.44 μm step size, 28 planes) were collected using the Cy5, Cy3, GFP and DAPI channels (center/band width, nm: excitation 545/39, 620/60, 470/40, 360/40, respectively; emission 605/75, 700/75, 525/45, 470/40, respectively). Stacked images were presented as the maximum intensity projection of the center five planes. All images were acquired with identical settings within each experiment. Cells that displayed oversaturated signal were excluded from analyses. For co-transfected conditions, only cells that were GFP positive and Cy3 positive were analyzed. The experimentalist was blinded to transfection conditions during the acquisition and data analyses for all the experiments described below.

### HEK293T N/C Ratio

The nuclear and cytoplasmic compartments were defined using DAPI and phalloidin fluorescent signals, respectively. Only cells with distinct and non-overlapping cytoplasmic and nuclear compartments were included in the analysis. The integrated fluorescence intensity of each fluorophore, corresponding to SOD1-myc or nanobody, was measured using a 2 μm×2 μm square region that was manually placed within the nucleus and cytoplasm of each cell. The square was placed in an area with signal that was representative of the overall compartment, thus avoiding areas of extreme bright or weak signal [67]. The N/C ratio for each fluorophore was calculated by dividing the intensity of the nuclear signal by the cytoplasmic signal. Cells from three random fields-of-view per condition were analyzed, for a total of 81-200 cells per condition over n=3 independent biological replicates.

### HEK293T Colocalization Analysis

The FIJI plug-in EzColocalization was used to obtain the Pearson correlation coefficient between colocalization of co-transfected SOD1-myc and nanobody signals (43). The phalloidin signal was used to define cell boundaries and manually create a whole cell outline of each cell included in the N/C ratio analyses described above. These whole cell outlines were then used as inputs to define the regions of interest for EzColocalization signal analysis.

### HEK293T Signal Intensity Analysis

The whole cell outlines described in the colocalization analysis were then used to measure the integrated fluorescence intensity of each cell using FIJI. The mean integrated fluorescence intensity of each fluorophore, corresponding to SOD1-myc or nanobody, was measured for each whole cell and plotted for analysis.

Western blot analysis: HEK293T cells transfected as described above in section 4.5 were washed with PBS and lysed in cold RIPA buffer (Westnet, BP-115-500) for 20 min on ice after which the lysates were clarified by centrifugation at 13,000 rpm for 15 min at 4° C. Protein concentration was estimated by bicinchoninic acid (BCA) assay (Thermo Scientific Pierce, 23227) and 20 μg of total protein was electrophoresed through 15% polyacrylamide gel and transferred onto PVDF membrane (Millipore, IPFL00010) for 1 h at 100V. The immunoblot was blocked for 1 h with blocking buffer (LICOR, 927-70001) and then incubated overnight at 4° C. with the primary antibodies: anti-myc (1:1000, DSHB 9E-10), anti-nanobody described above (1:2500), anti-tubulin (Sigma Aldrich, T5168, 1:5000), anti-GAPDH (Sigma Aldrich, G8795, 1:2000), anti-SOD1 (Abcam ab79390, 1:15000). Blots were probed for 1 h with IRDye conjugated secondary antibodies (LICOR) and imaged using the Odyssey Infrared Imager (LICOR, 9120).

For Western blot analysis of recombinant proteins, recombinant SOD1 WT and G93A were denatured by boiling in 1× Laemmli buffer (Westnet, BP-111R) and subjected to Western blot analysis as described above. The immunoblot was incubated overnight with Nb61 (0.2 μg/ml in PBS) at 4° C. followed by incubation with the rabbit anti-nanobody described above (1:2500) for 2 h at ambient temperature. As a positive control, a duplicate immunoblot was processed with a commercial pan anti-SOD1 antibody (Abcam ab79390, 1:15000). Blots were probed for 1 h with IRDye conjugated secondary antibodies (LICOR) and imaged as described above.

iPSC culture, motor neuron differentiation and lentiviral transduction: Human WT (1016a) and SOD1 A4V ALS-patient (39b) iPSCs were differentiated into motor neurons following previously established 3D methods and were dissociated and cryogenically stored at 21 days of differentiation. Thawed neurons were plated as single cells in 384-well cell culture plates previously coated with laminin (2.5 μg/mL) and fibronectin (7.5 μg/mL). Cells were plated in complete media, comprised of Neurobasal medium (Gibco, 21103-049), 1×N2 supplement (Gibco, 17502-048), 1×B27 supplement (Gibco, 17504044), 1× Glutamax (Gibco, 35050061), 1× non-essential amino acids (Gibco, 11140-050), 1× penicillin-streptomycin (Gibco, 10378-016), 3.2 mg/mL D-glucose, 20 μM ascorbic acid, 10 ng/mL brain-derived neurotrophic factor (BDNF), 10 ng/mL ciliary neurotrophic factor (CNTF), and 10 ng/ml glial cell-derived neurotrophic factor (GDNF).

Plasmids for lentiviral transduction are described above under ‘Plasmid construction’, Lentiviral particles were prepared for GFP alone (negative control), Nb61 and Nb61-PEST at the Viral Vector Core of the Gene Therapy Center within UMass Chan Medical School. Note that GFP is expressed as a reporter, and not a fusion protein, via these constructs. At the time of plating (8000 cells per well), lentiviral particles were added to the complete media together with 5 μg/mL polybrene for a final viral titer of 10⁸ vp/mL and an estimated MOI of 125. For all experiments, lentivirus expressing GFP alone, Nb61 and Nb61-PEST were tested in parallel, within the same plate. At day in vitro (DIV) 7, plates were fixed with 4% paraformaldehyde for 15 minutes at ambient temperature for staining and immunolabeling as described below. For viral transduction efficiency calculations, a subset of wells underwent live-cell imaging with Hoechst (nuclear stain, all cells) and propidium iodide (dead cells) at DIV7. Transduction efficiency was calculated as the number of GFP positive cells divided by the total number of live cells per well across n=3 wells.

Immunofluorescence microscopy analysis of iPSC-derived motor neurons: Fixed cells within 384-well plates were blocked (5% FBS, 2% BSA, 0.1% Triton X100 in PBS) and incubated with neurofilament H (NFH) clone SMI-32 (1:1000, Biolegend, 801701) and SOD1 (1:500, Enzo, ADI-SOD-100-J) primary antibodies overnight, washed, then incubated with animal matched Alexa-conjugated secondaries and Hoechst counterstain. Plates were imaged on an ImageXpress Pico System (Molecular Devices) using automated capture. Image analysis was performed in MetaXpress (Molecular Devices, version 6.6.2.46). Technical replicates were defined as an individual well of a 384-well plate. In-plate technical replicates ranged from 4 to 6 wells per condition. Biological replicates were defined as independent experiments from a separate thaw of iPSC-derived motor neurons. Herein, three biological replicates of SOD1 A4V and two of WT motor neurons were analyzed. Additionally, both genotypes of neurons originated from two independent differentiation batches. In the figures, defined symbols denote a specific biological replicate as defined in the figure legend.

### Neurite Tracing Analysis

Images were acquired at 10× in a Pico high content imager (Molecular Devices) and stitched to create one image containing three fields of view across the well of a 384-well plate. Stitched images were then analyzed with a custom neurite tracing script written in the MetaXpress image analysis software (Molecular Devices, version 6.6.2.46). In brief, neurite detection was set to be ≥3 times the intensity of the background, to be in the width range of 0-5 μm, and be at least 2 μm long to be counted. All calculated lengths were summed across the stitched fields covering approximately 50% of the area of a well of a 384-well plate. The analyst was blinded to the conditions.

### SOD1 Intensity Analysis

Images were acquired at 10× in a Pico high content imager (Molecular Devices) and stitched as described for neurite tracing. Stitched images were then analyzed with a custom script written in the MetaXpress image analysis software (Molecular Devices, version 6.6.2.46) as follows. GFP positive cells were identified using the “auto-threshold” tool in the GFP channel. The threshold was set to ≥3 times the background intensity. The resulting GFP positive area mask was then overlaid on the SOD1 channel (i.e., to measure SOD1 intensity only in cells with positive GFP signal, and therefore transduced with the lentiviral constructs). The SOD1 intensity corresponding to the GFP positive mask was calculated as a total intensity value for each well. The total intensity for each well was then divided by the total area of the GFP+ mask to control for different numbers and different sizes of GFP positive cells. Cells expressing Nb61 or Nb61-PEST were compared to cells expressing GFP alone (n=3 technical replicates) using GraphPad Prism (v9.3). The analyst was blinded to the conditions.

Differential scanning fluorimetry (DSF): DSF measurements were performed similarly to our previous report [48]. SOD1 A4V (10 UM, monomeric concentration), Nb61 (10 μM), Nb54 (10 μM), or mixtures of SOD1 A4V and nanobody (10 UM each) were prepared in PBS and incubated on ice for 1 h. SYPRO Orange (Invitrogen #S6651) was then added with a final concentration of 25× with a total reaction volume of 20 uL. All samples were run in duplicate in 384-well plates. The dye diluted in PBS containing no protein served as a negative control. Thermal scanning and fluorescence measurements were performed with a Bio-Rad C1000 Touch Thermal Cycler with CFX384 Optical Reaction Module (Bio-Rad #1845384). The samples were gradually heated at 0.3° C./5 sec. Fluorescence measurements were acquired with each temperature increment. The fluorescence intensities from the dye-only reactions were subtracted from the experimental wells. The resulting fluorescence intensities from duplicate experimental wells were averaged and normalized to 1 (i.e., each experimental curve was normalized separately to the highest fluorescence intensity measurement within that curve) and plotted as a function of temperature. The temperature corresponding to the maxima of the first derivative of each melting transition was used as an estimate of the melting temperature (Tm). All data are representative of at least three independent biological experiments.

Statistical Analysis: Statistical analysis was performed using Graphpad Prism 9 (v9.3). Kruskal-Wallis with Dunn's multiple comparison test was used for all HEK293T and iPSC-derived motor neuron experiments, with the exception of signal intensity linear regression analysis. Two-way ANOVA with Dunnett's multiple comparison test was used for the ELISA experiments. A p-value less than 0.05 was considered significant.

### Example 1: Nanobodies with Selectivity for ALS-Linked SOD1

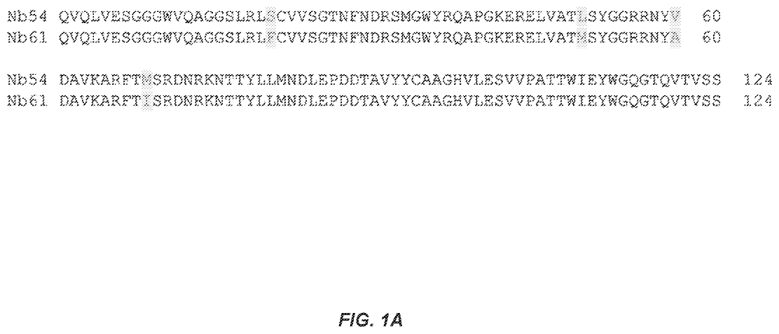

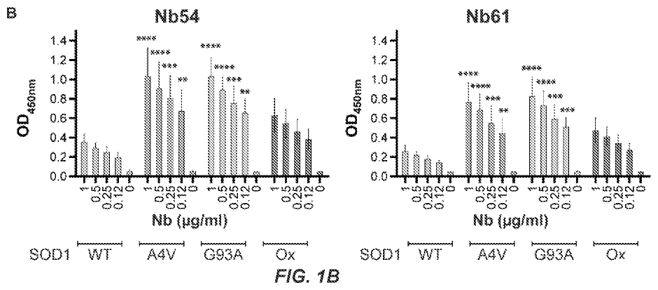

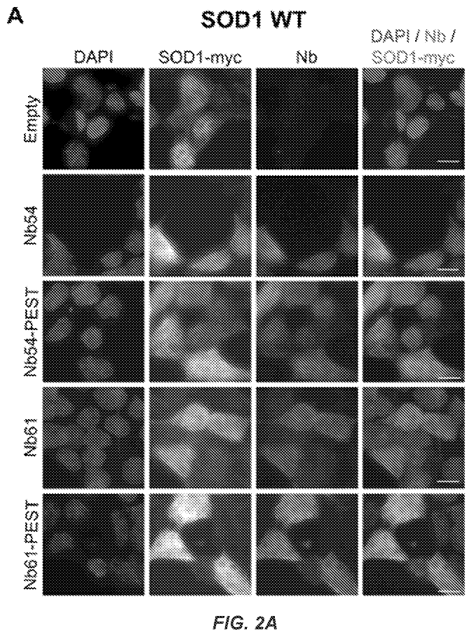

We sought to identify anti-SOD1 nanobodies that exhibit selectivity for mutant and misfolded forms of SOD1, as such biologics have therapeutic potential. Llamas were immunized simultaneously with recombinant SOD1 WT, SOD1 G93A and an oxidized form of SOD1 (SODox) that we and others have shown to adopt a mutant-like, misfolded conformation [10]. Blood from the immunized animals was used for single domain antibody, or nanobody, library construction and binder discovery to identify clones that bind SODox over SOD1 WT. Two nanobodies with 96.7% sequence identity, referred to as Nb54 and Nb61, were found to exhibit >5-fold selectivity for SODox over the native SOD1 WT protein in this screen (FIG. 1A). We then expressed and purified Nb54 and Nb61 as recombinant proteins from E. coli for further validation and characterization.

We first assessed the reactivity of Nb54 and Nb61 for recombinant SOD1 proteins by an enzyme linked immunosorbent assay (ELISA). In addition to the SOD1 variants used as immunogens to create these anti-SOD1 nanobodies (e.g., SOD1 WT, SOD1 G93A and SODox), we assessed reactivity to SOD1 A4V, representing the most common and aggressive variant in the North American ALS patient population [39]. Relative to SOD1 WT, both Nb54 and Nb61 exhibited 3-4-fold higher reactivity toward SOD1 A4V and SOD1 G93A when tested with 0.12-1 μg/ml concentrations of the respective Nb (FIG. 1B). Nb61 also reacted with the denatured form of both SOD1 WT and G93A (FIG. S1). Both Nb54 and Nb61 tended to exhibit higher reactivity toward SODox compared to SOD1 WT, however, this difference in reactivity did not reach statistical significance. Given that SOD1 A4V was not used as an immunogen for the generation of these nanobodies, the high reactivity of Nb54 and Nb61 for SOD1 A4V reinforces the notion that ALS-linked SOD1 variants share a common misfolded conformation [6,40].

### Example 2: Anti-SOD1 Nanobodies Lead to Enhanced, Rather than Reduced, Levels of Ectopic SOD1 in Cellulo

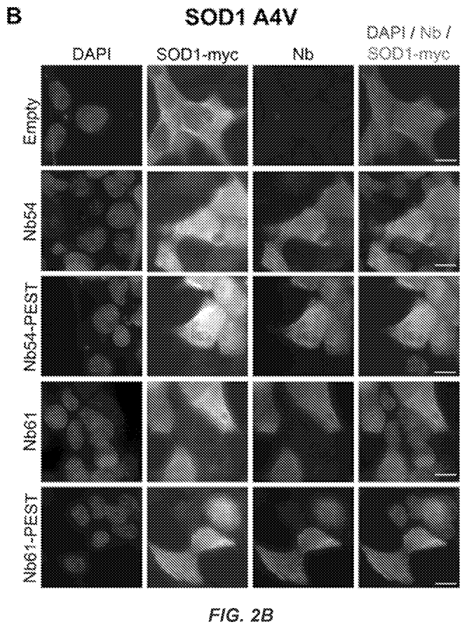

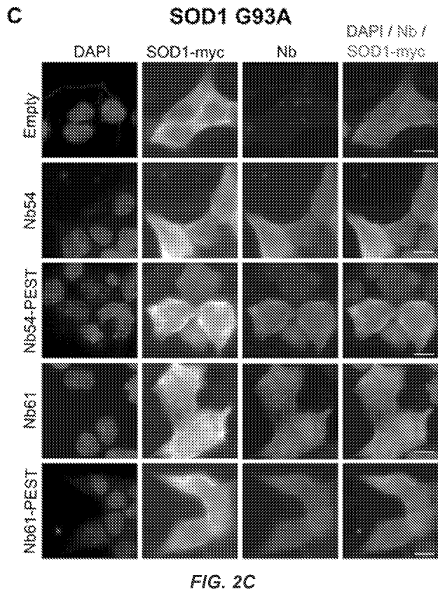

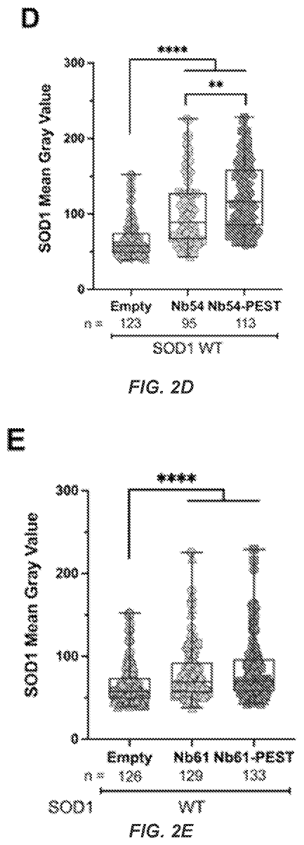

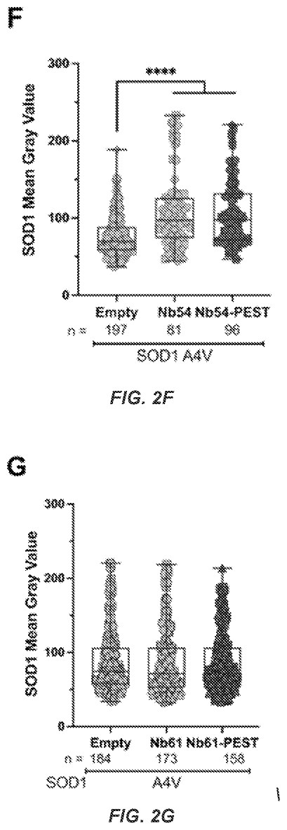

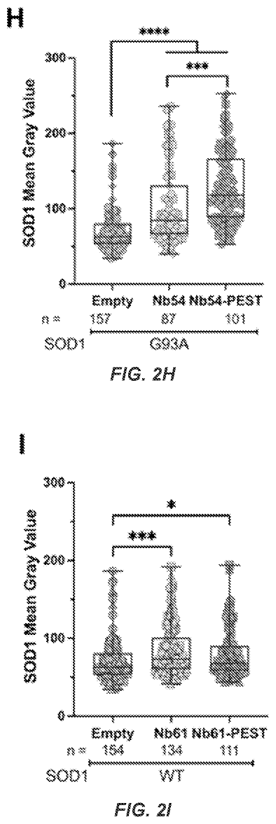

Intracellular clearance of nanobody-bound antigen generally does not occur in the absence of a proteolytic targeting signal, such as the PEST degron. PEST sequences are rich in proline, glutamate, serine and threonine residues, and are found in proteins with particularly short half-lives [41]. Fusion of PEST sequences to proteins, including antibody fragments that recognize neurogenerative-disease associated proteins a-synuclein [35,42,43] and Huntingtin [44], induce their degradation through the ubiquitin proteasome pathway. Here, we engineered versions of both Nb54 and Nb61 with a C-terminal PEST sequence. We initiated characterization of the SOD1/Nb interaction in cellulo using HEK293T cells, as this represents a tractable cell line with high transfection efficiency. To test whether the nanobodies could induce degradation of ALS-linked SOD1 variants, HEK293T cells were co-transfected with either myc-tagged SOD1 WT (FIG. 2A), SOD1 A4V (FIG. 2B) or SOD1 G93A (FIG. 2C), together with either nanobody, nanobody-PEST, or an empty control plasmid (i.e., the nanobody plasmid without the nanobody gene; FIG. 2A-I). Cells were stained 24h post transfection with anti-nanobody and anti-myc antibodies for detection of nanobody and ectopic SOD1-myc (WT, A4V, and G93A), respectively (FIG. 2A-C). As the fluorescent signal intensity is indicative of protein abundance, we measured the mean gray values or fluorescence intensities corresponding to SOD1-myc and nanobody on a per cell basis. Co-transfection of either Nb54 or Nb54-PEST resulted in significantly higher SOD1-myc signal intensities for all SOD1 variants relative to control cells that were co-transfected with that SOD1 variant and the empty vector (FIG. 2D, F, H). Similar results were obtained from cells co-transfected with SOD1 WT and Nb61 or Nb61-PEST (FIG. 2E). SOD1 A4V-expressing cells co-transfected with Nb61 or Nb61-PEST showed no difference in SOD1-myc signal intensity compared to cells co-expressing SOD1 A4V and the empty vector (FIG. 2G). While SOD1 G93A-expressing cells co-transfected with Nb61 or Nb61-PEST demonstrated a significant increase in SOD1 G93A signal intensity, the effect was more robust with Nb54 (FIG. 2I).

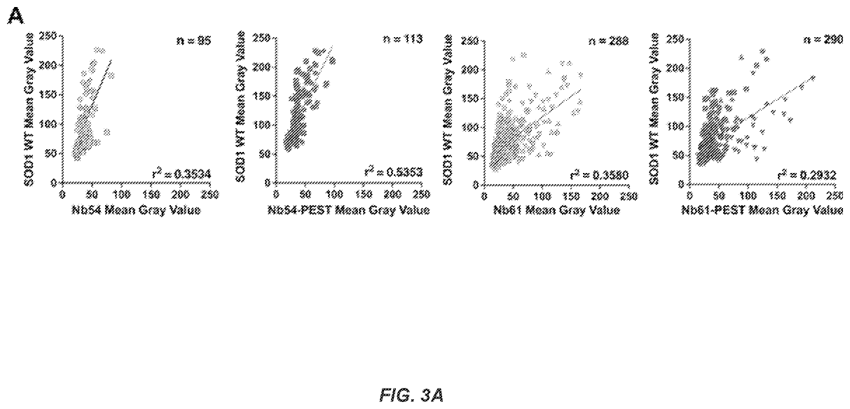

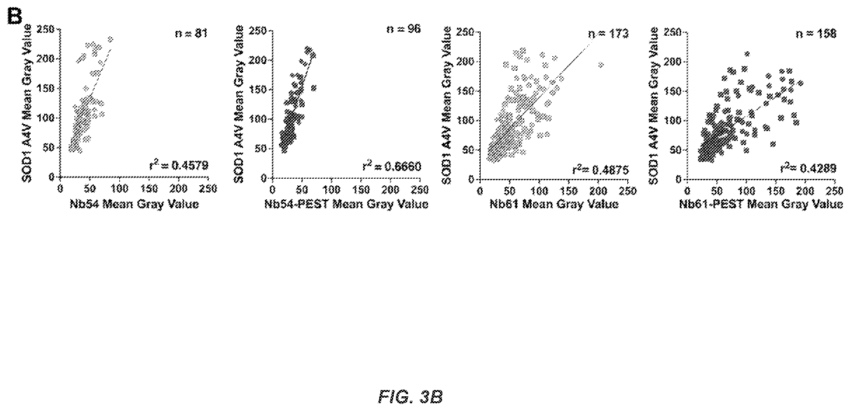

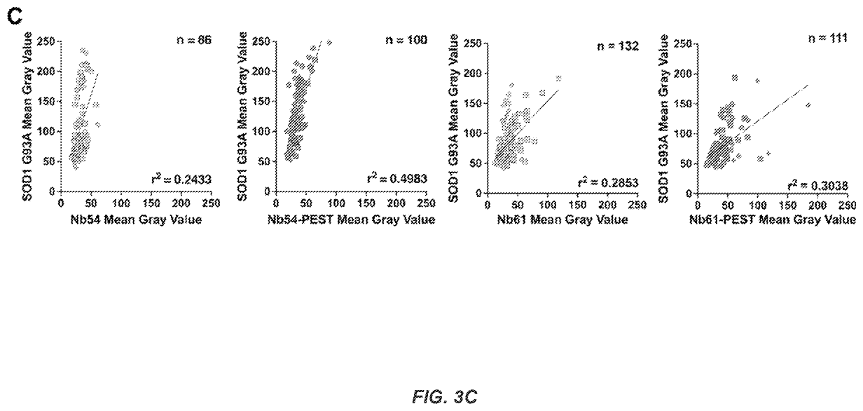

We also examined the SOD1-myc levels by Western blot analysis of the cell lysates from the HEK293T co-transfection experiments (FIG. S2A). In contrast to the per cell fluorescence intensity analyses (FIG. 2), the outcomes of the lysate-based Western blot analyses were variable among experiments (FIG. S2B), likely due to the variation of transgene expression across a population of cells that have undergone transient co-transfection. To examine this further, we performed a linear regression analysis of fluorescence intensity corresponding to anti-myc versus anti-Nb for cells co-expressing either myc-tagged SOD1 WT (FIG. 3A), SOD1 A4V (FIG. 3B) or SOD1 G93A (FIG. 3C) with the various nanobody constructs. For all SOD1-myc and nanobody comparisons, including nanobody-PEST constructs, there was a positive correlation between SOD1-myc fluorescence intensity and nanobody fluorescence intensity on a per cell basis (FIG. 3), indicating that the nanobodies generally enhance SOD1-myc expression.

In sum, the outcomes of the fluorescence intensity analyses are consistent with an association between SOD1-myc and nanobody proteins in cellulo. Unexpectedly, the PEST sequence was ineffective at targeting mutant SOD1 to the proteasome, as SOD1 signal intensities generally increased (rather than decreased) upon co-expression with nanobody-PEST constructs (FIGS. 2 and 3). These observations suggest that the PEST sequences are not exposed or functional when fused to these anti-SOD1 nanobodies.

### Example 3: The Subcellular Localization of Mutant SOD1 is Restored by Co-Expression of Anti-SOD1 Nanobodies

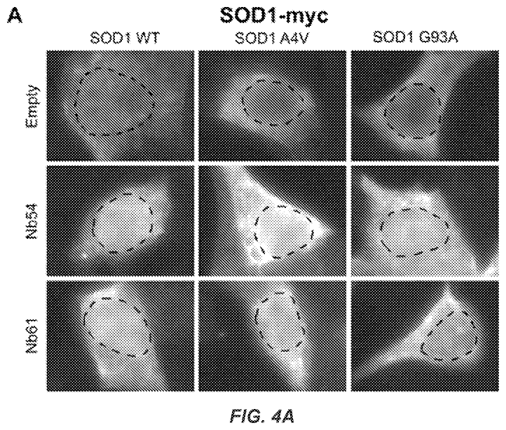

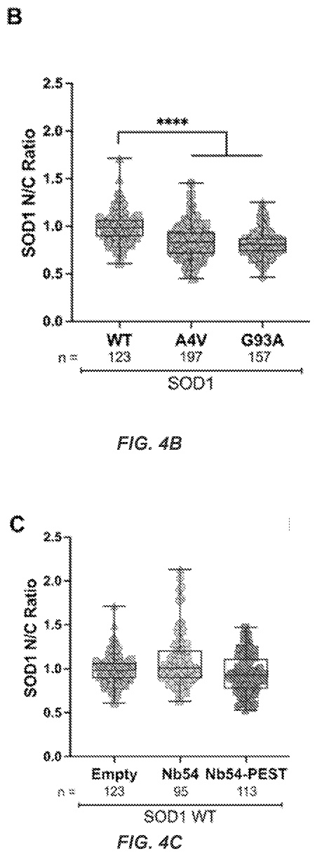

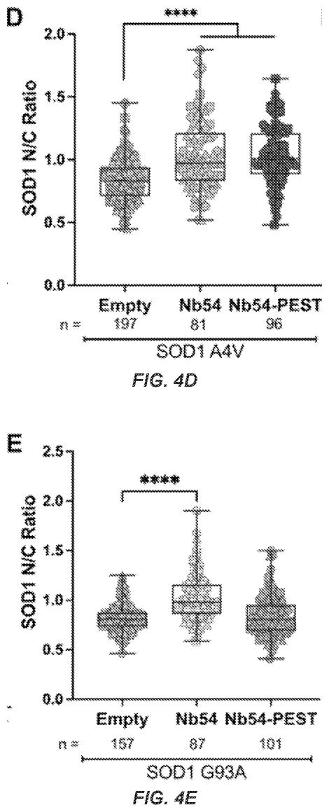

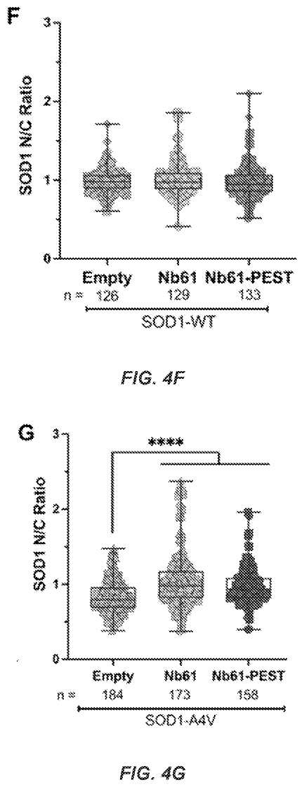

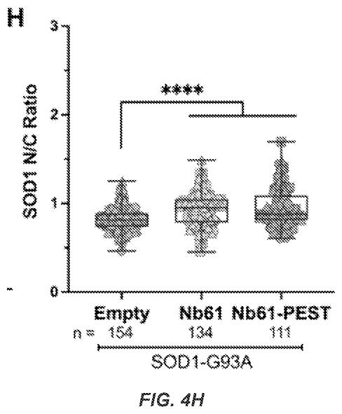

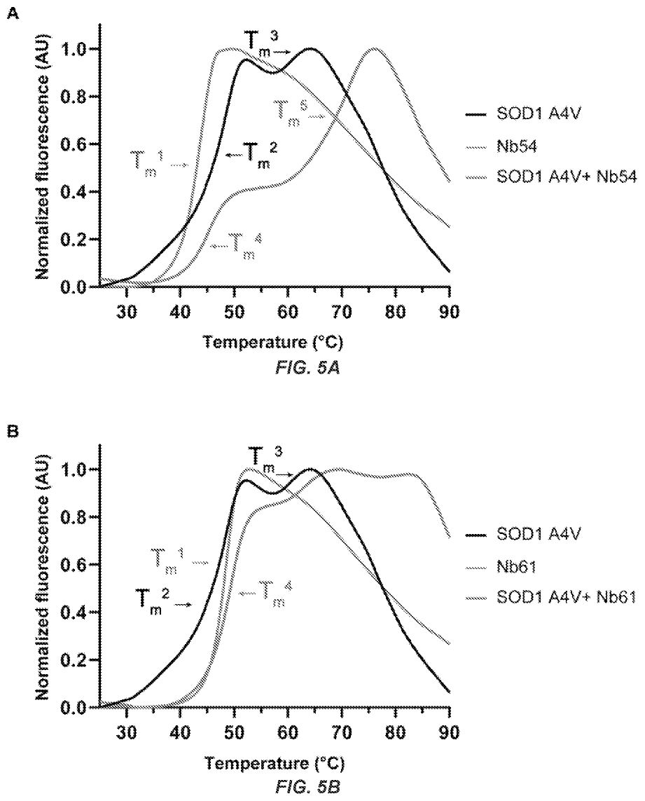

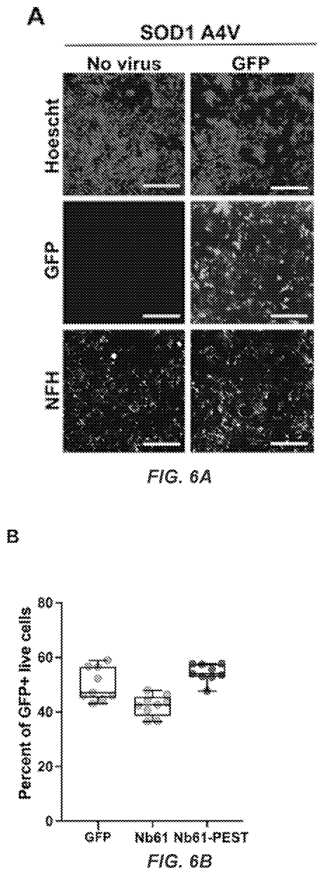

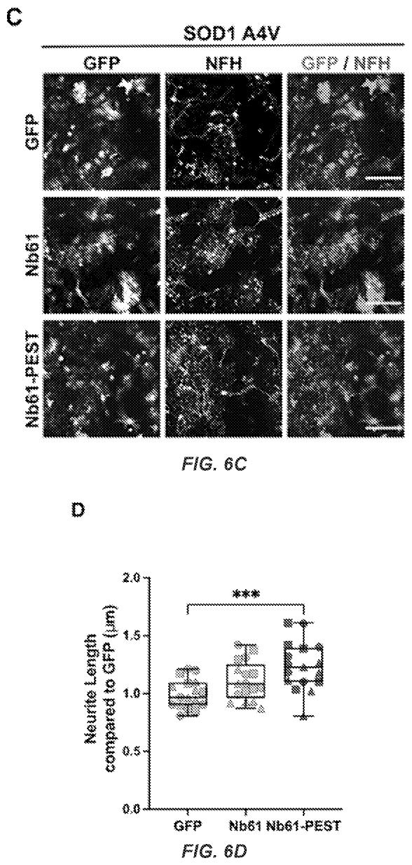

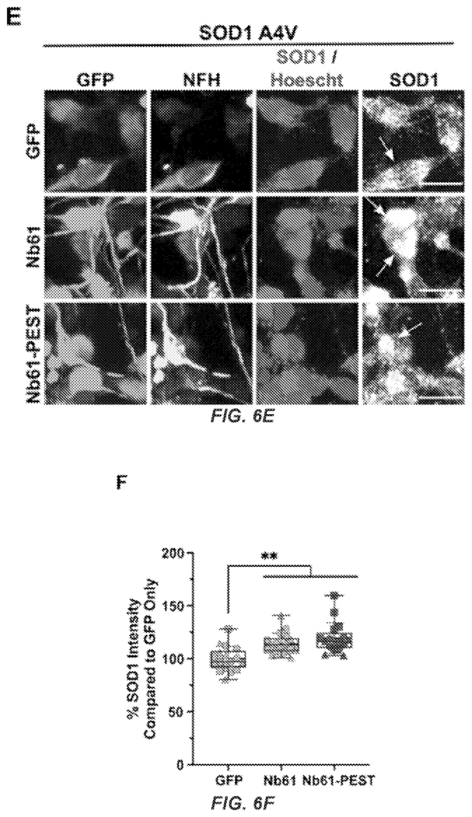

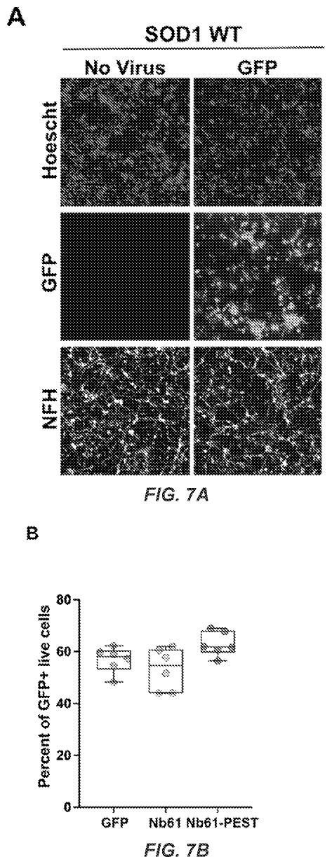

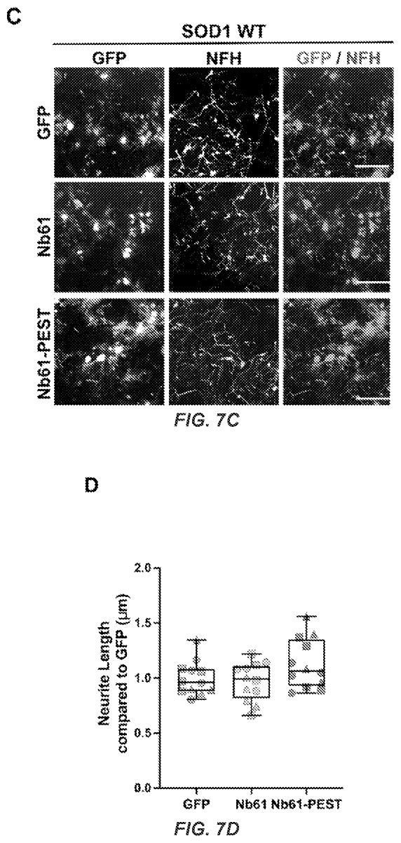

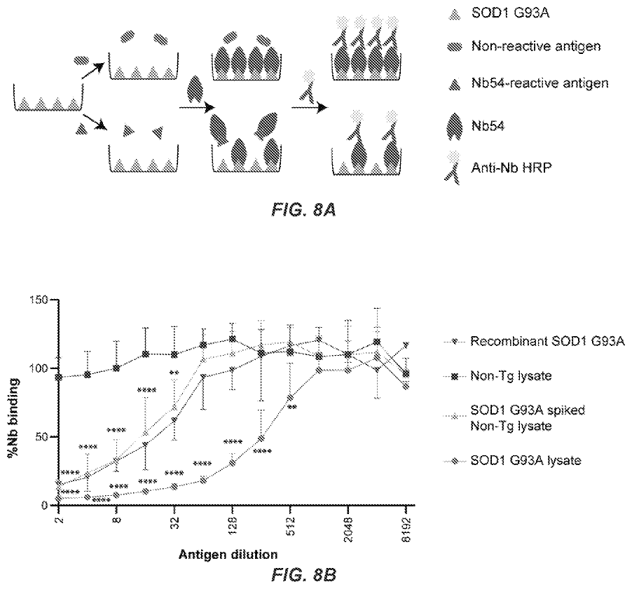

The fluorescence intensity analyses also revealed myc-tagged SOD1 WT expression in both the nucleus and cytoplasm, consistent with previous reports of subcellular SOD1 localization in mammalian cells and nervous tissue [2,45]. Conversely, SOD1 A4V and SOD1 G93A expression were more cytoplasmic relative to SOD1 WT (FIG. 4A), possibly due to a misfolded conformation that favors cytoplasmic SOD1 localization [11,23]. To quantify this phenotype, integrated fluorescence signal intensities of SOD1-myc were measured in the nucleus (defined by DAPI) and the cytoplasm (defined by phalloidin), and nuclear to cytoplasmic ratios (N/C) were determined for each condition. N/C measurements were significantly lower for SOD1 A4V and G93A compared to SOD1 WT, which exhibited a mean N/C of ˜1, indicating similar levels of SOD1 WT in the nucleus and cytoplasm (FIG. 4B). While co-transfection of Nb54 or Nb54-PEST did not affect the N/C of SOD1 WT (FIG. 4C), these nanobodies resulted in a significant increase in the N/C to a mean of ˜1 for SOD1 A4V (FIG. 4C,D). The N/C for SOD1 G93A was likewise increased to a mean of ˜1 with co-expression of Nb54, although not with Nb54-PEST (FIG. 4E). Similarly, co-expression of Nb61 and Nb61-PEST did not affect the N/C of SOD1 WT (FIG. 4F), but these nanobodies significantly increased the N/C for both SOD1 A4V (FIG. 4G) and SOD1 G93A (FIG. 4H).

### Example 4: Anti-SOD1 Nanobodies Stabilize Mutant SOD1 In Vitro

Collectively, the co-transfection studies in HEK293T cells are consistent with an association between mutant SOD1 and our anti-SOD1 nanobodies in cellulo. These results also imply that nanobody binding induces a conformational change within mutant SOD1 that favors a WT-like localization in cells. As ALS-linked SOD1 variants adopt a misfolded and thermodynamically destabilized conformation [5-7,40,46,47], we investigated the effects of nanobody binding on mutant SOD1 thermal stability. To this end, we employed differential scanning fluorimetry (DSF), a technique that we and others have used to study the stability of recombinant misfolded proteins [48], including ALS-linked SOD1 [49]. With this thermal shift assay, protein unfolding is monitored using the SYPRO orange dye, which reports on the exposure of hydrophobic regions [50,51]. Thermal denaturation of SOD1 A4V resulted in two melting transitions with a melting temperature (Tₘ) of 48.9° C. and 60° C., respectively (FIG. 5), consistent with two differentially metalated SOD1 A4V species with distinct melting temperatures [46]. A Tₘ of 43.8° C. and 48.6° C. was determined for Nb54 (FIG. 5A) and Nb61 (FIG. 5B), respectively. Co-incubation of SOD1 A4V and Nb54 resulted in a DSF curve that was substantially shifted to the right. Further, there was a new melting transition with a Tₘ of 71.1° C., indicative of an SOD1 A4V/Nb54 complex with enhanced stability relative to either protein alone. The DSF curve for the SOD1 A4V/Nb54 complex contained another melting transition with a Tₘ 45° C., which likely represents unbound SOD1 A4V and/or unbound Nb54 (FIG. 5A). The DSF curve for the SOD1 A4V/Nb61 complex was also shifted toward higher temperatures (FIG. 5B). While the individual melting transitions were not well resolved in the case of SOD1 A4V/Nb61, the DSF curve also contained a peak at a high temperature (˜85° C.), consistent with a melting transition for SOD1 A4V/Nb61 with enhanced thermostability. In sum, these data indicate that complex formation between anti-SOD1 nanobodies and SOD1 A4V have a stabilizing effect on the mutant protein. A DSF analysis for SOD1 WT was not pursued due to the high thermostability (Tₘ>90° C.) of this protein, also reported by others [46,49].

### Example 5: Anti-SOD1 Nanobody Expression is Non-Toxic and Induces Enhanced Neurite Outgrowth in Human SOD1 A4V Motor Neurons

Aiming to study our nanobodies in a disease relevant context without SOD1 overexpression, we generated lentiviral particles for transduction and expression of Nb61, Nb61-PEST or GFP in human iPSC-derived motor neurons. Virus expressing GFP served as a negative control, as all of the lentiviral constructs were designed to co-express GFP from an internal ribosome entry site (IRES) for identification of transduced cells. Lentiviral particles were delivered to human SOD1 A4V neurons (FIG. 6A), which reportedly exhibit reduced cell health compared to control lines without SOD1 mutations [52,53]. Lentiviral transduction efficiencies were typically similar (40-60%) across conditions (FIG. 6B). Seven days post viral transduction, we assessed neuronal health by comparing total neurite lengths (anti-NFH) between constructs (FIG. 6C) [53]. Compared to SOD1 A4V neurons expressing the GFP control lentivirus, SOD1 A4V neurons expressing Nb61 and Nb61-PEST exhibited a greater total neurite length, which reached statistical significance with Nb61-PEST (FIG. 6D). SOD1 A4V neurons were also stained with anti-SOD1, allowing for quantification of endogenous SOD1 fluorescence intensity within transduced GFP-positive cells (FIG. 6E). Expression of either Nb61 or Nb61-PEST resulted in higher SOD1 signal intensities in SOD1 A4V neurons (FIG. 6F). These results are consistent with the enhanced SOD1-myc fluorescence intensities observed under most conditions in HEK293T cells upon co-expression of anti-SOD1 nanobodies (FIG. 2). Whether there are differences in N/C between conditions is unknown, as occurrences of cell clumping and the presence of non-neuronal cells (that also express SOD1) in iPSC-derived neuronal cultures precluded a rigorous N/C analysis as we showed for HEK293T cells.

We also transduced SOD1 WT neurons with our set of lentiviral constructs, which were generated and handled using the same procedures and reagents as for SOD1 A4V neurons above [54]. Consistent with other reports [52,53], we routinely observed that the viability and neurite outgrowth of SOD1 WT neurons is greater than SOD1 A4V neurons, which is reflected by the differences in cell and neurite densities of the respective neuronal lines upon thawing (compare FIGS. 6A and 7A). As for SOD1 A4V lines, lentiviral transduction efficiency of SOD1 WT neurons was similar across all conditions (FIG. 7B). Compared to the GFP control, neurite length was not significantly affected by expression of Nb61 or Nb61-PEST (FIG. 7C,D). Therefore, expression of anti-SOD1 Nb61 was not toxic to human neurons under these conditions, but instead conferred a health benefit to the SOD1 A4V line.

### Example 6: Anti-SOD1 Nanobodies Detect Human SOD1 G93A in Lysates from an ALS Mouse Model

SOD1-G93A transgenic rodent models are used most for preclinical testing of SOD1-based therapeutics in the ALS field ([4]; [14]). To investigate the preclinical therapeutic utility of our anti-SOD1 nanobodies, we tested whether Nb54 could detect ectopic human SOD1 G93A in lysates prepared from SOD1^{G93A} mouse spinal cord tissue with a competitive ELISA as follows. Recombinant SOD1 G93A was coated onto wells of the ELISA plate, and the binding of Nb54 to the immobilized SOD1 G93A was measured as a function of increasing amounts of a “competing” antigen (FIG. 8A). In the absence of SOD1^{G93A} lysate or an otherwise Nb54-reactive antigen, maximal binding of Nb54 to the immobilized SOD1 G93A is expected (FIG. 8A; top). In the presence of a competing antigen that binds Nb54, there is less available Nb54 to react with the immobilized SOD1 G93A in the ELISA plate, and thus reduced signal in the assay (FIG. 8A; bottom). As Nb54 binds to recombinant SOD1 G93A, increasing concentrations of this antigen was used as a positive control to verify competition with Nb54 and a reduced signal in the ELISA (FIG. 8B). In contrast, spinal cord lysates derived from non-transgenic (Non-Tg) animals were unable to compete in this assay, even at the highest concentration tested, indicating a lack of reactivity between Nb54 and endogenous murine SOD1 WT. However, the same Non-Tg lysates spiked with recombinant SOD1 G93A did compete with Nb54/immobilized SOD1 G93A binding. Importantly, SOD1^{G93A} spinal cord lysates effectively competed with Nb54/immobilized SOD1 G93A binding in a dose-dependent manner, indicating that Nb54 reacts with ectopic human SOD1 G93A in this lysate (FIG. 8B). These results demonstrate target engagement between Nb54 and human SOD1 G93A in a complex biological mixture and indicate there is no cross-reactivity between Nb54 and marine SOD1 WT, thus providing a foundation for future preclinical investigation of Nb54 in SOD1^{G93A} mice.

### DISCUSSION

In this study, we developed and characterized two anti-SOD1 nanobodies, Nb54 and Nb61, as potential therapeutic molecules for ALS. Nanobodies can be engineered to direct their cognate antigens to different cellular machineries, thereby serving as versatile tools for managing intracellular SOD1 [35]. Contrary to our initial hypothesis, the addition of a PEST tag to Nb54 or Nb61 did not result in reduced SOD1 levels. This may be due to changes in structural properties of the PEST tag, such as poor solvent accessibility, upon fusion with Nb54 and Nb61. Different outcomes may be achieved by engineering a spacer sequence between the nanobody and PEST sequences and/or placing the PEST sequence at the N-terminus (as opposed to the C-terminus herein). Irrespective of the presence of PEST tag, both Nb54 and Nb61 enhanced SOD1-myc signal intensities in HEK293T cells. In contrast, no changes in SOD1 levels were observed with anti-SOD1 nanobodies from a different source [55]. In the case of Nb54-PEST, signals for myc-tagged SOD1 WT and G93A were higher in the co-transfection studies compared to nanobody without PEST. Similarly, the effects of Nb61-PEST were more pronounced than Nb61 in human SOD1 A4V neurons. Therefore, the PEST sequence may also induce structural changes within the nanobodies that in turn impact the Nb/SOD1 interaction. These results highlight the potential for further optimization of these anti-SOD1 nanobodies for SOD1 target engagement.

Anti-SOD1 nanobodies also affected the nucleocytoplasmic distribution of mutant SOD1 proteins in HEK293T cells. Unlike SOD1 WT, which is expressed in both the nucleus and cytoplasm, ALS-linked SOD1 mutants exhibit enhanced cytoplasmic localization that was observed here and reported previously by others [11,23,56,57]. Cytoplasmic localization of mutant SOD1 is likely a result of mutation-induced misfolding, which could expose a putative nuclear export signal and thus nuclear export of mutant SOD1 via CRM1 (Chromosomal Maintenance 1) [23]. Co-expression of our anti-SOD1 nanobodies restored mutant SOD1 in the nucleus to SOD1 WT levels. This appears to be a unique property of our nanobodies that was not reported for other anti-SOD1 intrabodies [32,33,55]. In this regard, the activity of our nanobodies may resemble the macrophage migration inhibitory factor, a chaperone-like protein that also restores the N/C of mutant SOD1 [56]. It is unlikely that Nb54 or Nb61 sequesters SOD1 within the nucleus, as both nanobodies are expressed throughout the nucleus and cytoplasm (FIG. 2). Rather, we speculate that binding of Nb54 or Nb61 to mutant SOD1 converts misfolded SOD1 into a more SOD1 WT-like conformation, thereby favoring the nuclear localization observed for SOD1 WT. This model is supported by the outcomes of our DSF studies, which revealed that both Nb61 and Nb54 exert a stabilizing effect when in complex with SOD1 A4V.

We noted some differences in the properties of our anti-SOD1 nanobodies when assessed in vitro as recombinant proteins by ELISA versus when expressed in cellulo. For example, Nb54 and Nb61 exhibited similar reactivities for SOD1 A4V and G93A in the ELISA, whereas Nb54 exerted a more pronounced effect on SOD1 signal intensities and mutant SOD1 N/C localization in HEK293T cells relative to Nb61. These results suggest that the physicochemical properties of the nanobodies and/or their capacity to interact with SOD1 proteins is influenced by additional factors in cellulo. Further, Nb54 and Nb61 were selective for both SOD1 A4V and SOD1 G93A over SOD1 WT in vitro, but appeared to engage with and enhance levels of myc-tagged SOD1 WT when co-expressed in HEK293T cells. One explanation could be that Nb/SOD1 WT interactions are facilitated by the over-expression conditions used for co-transfection studies. We expect that some degree of anti-SOD1 nanobody target engagement with SOD1 WT will not preclude their therapeutic utility, as ectopic Nb61 expression was not toxic to human neurons expressing SOD1 WT or mutant SOD1 A4V. Further, enhancement of SOD1 WT levels may be preferred and possibly beneficial over a reduction in SOD1, particularly in a disease context for which there is elevated oxidative stress [15] [5] [24] [3,58,59]. Additionally, a beneficial effect has been observed in ALS-SOD1 models upon treatment with diacetyl-bis(4-methylthiosemicarbazonato) copper(II) [Cu(II)(atsm)], which promotes metalation and increases the levels of SOD1 [60-62]. Thus, anti-SOD1 nanobodies offer an alternative approach to current gene silencing strategies that target both mutant and WT SOD1 alleles, which causes an overall reduction in SOD1.

To assess the potential of our anti-SOD1 proteins for preclinical studies and eventual therapeutic application, we performed studies in ALS-relevant models including human iPSC-derived motor neurons harboring the SOD1 A4V mutation. SOD1 A4V is the most common and aggressive ALS-linked mutation in North America, and therefore biologics targeting this protein are expected to have high therapeutic value for the ALS field. Although SOD1 A4V was not used as an antigen for our anti-SOD1 nanobodies, Nb54 and Nb61 exhibited selectivity for this mutant protein. This observation raises the possibility that our anti-SOD1 nanobodies could be reactive for other SOD1 variants, of which there are over 170 reported to date [63]. Transduction of lentiviruses expressing Nb61 and Nb61-PEST were not toxic to human neurons, but rather conferred a beneficial effect on the health of SOD1 A4V neurons. Both neurite outgrowth and SOD1 levels were enhanced upon expression of Nb61 compared to the control condition. In addition to human neurons, we also assessed the utility of our anti-SOD1 nanobodies for preclinical studies using the SOD1^{G93A} mouse model [4]. Our results demonstrate that Nb54 binds human SOD1 G93A in the context of mouse spinal cord lysate. Together, these proof-of-concept studies warrant future investigations with a larger panel of human neurons harboring different ALS-linked SOD1 mutations and a cohort of SOD1^{G93A} mice to further assess the therapeutic potential of anti-SOD1 nanobodies for ALS.

In sum, the nanobodies developed and characterized herein appear to stabilize the physiological conformation of SOD1. This stabilization may underlie the restoration of mutant SOD1 to normal subcellular locations in immortalized cells and confer protection in otherwise unhealthy human ALS SOD1 A4V neurons. Given that mutant SOD1 instability appears to correlate with ALS disease severity in humans [7], we propose that boosting levels of functional and natively folded SOD1 with anti-SOD1 nanobodies is a viable therapeutic direction for treating ALS.

### REFERENCES

1. Brown, R. H.; Al-Chalabi, A. Amyotrophic Lateral Sclerosis. N Engl J Med 2017, 377, 162-172, doi:10.1056/NEJMra1603471.
2. Pardo, C. A.; Xu, Z.; Borchelt, D. R.; Price, D. L.; Sisodia, S. S.; Cleveland, D. W.; Brown, R.; Price, D.; Sisodia, S.; Cleveland, D.; et al. Superoxide dismutase is an abundant component in cell bodies, dendrites, and axons of motor neurons and in a subset of other neurons. Proceedings of the National Academy of Sciences 1995, 92, 954-958, doi:10.1073/pnas.92.4.954.
3. Trist, B. G.; Hilton, J. B.; Hare, D. J.; Crouch, P. J.; Double, K. L. Superoxide Dismutase 1 in Health and Disease: How a Frontline Antioxidant Becomes Neurotoxic. Angew Chem Int Ed Engl 2021, 60, 9215-9246, doi:10.1002/anie.202000451.
4. Gurney, M. E.; Pu, H.; Chiu, A. Y.; Dal Canto, M. C.; Polchow, C. Y.; Alexander, D. D.; Caliendo, J.; Hentati, A.; Kwon, Y. W.; Deng, H. X.; et al. Motor neuron degeneration in mice that express a human Cu,Zn superoxide dismutase mutation. Science 1994, 264, 1772-1775, doi:10.1126/science.8209258.
5. Rotunno, M. S.; Bosco, D. A. An emerging role for misfolded wild-type SOD1 in sporadic ALS pathogenesis. Frontiers in Cellular Neuroscience 2013, 7, 1-16, doi:10.3389/fncel.2013.00253.
6. Rotunno, M. S.; Auclair, J. R.; Maniatis, S.; Shaffer, S. A.; Agar, J.; Bosco, D. A. Identification of a misfolded region in superoxide dismutase 1 that is exposed in amyotrophic lateral sclerosis. Journal of Biological Chemistry 2014, 289, 28527-28538, doi:10.1074/jbc.M114.581801.
7. Wang, Q.; Johnson, J. L., Agar, N. Y.; Agar, J. N. Protein aggregation and protein instability govern familial amyotrophic lateral sclerosis patient survival. PLOS Biol 2008, 6, e170, doi:10.1371/journal.pbio.0060170.
8. Medinas, D. B.; Rozas, P.; Martinez Traub, F.; Woehlbier, U.; Brown, R. H.; Bosco, D. A.; Hetz, C. Endoplasmic reticulum stress leads to accumulation of wild-type SOD1 aggregates associated with sporadic amyotrophic lateral sclerosis. Proceedings of the National Academy of Sciences 2018, 115, 201801109, doi:10.1073/pnas.1801109115
9. Guareschi, S.; Cova, E.; Cereda, C.; Ceroni, M.; Donetti, E.; Bosco, D. A.; Trotti, D.; Pasinelli, P. An over-oxidized form of superoxide dismutase found in sporadic amyotrophic lateral sclerosis with bulbar onset shares a toxic mechanism with mutant SOD1. Proceedings of the National Academy of Sciences of the United States of America 2012, 109, 5074-5079, doi:10.1073/pnas.1115402109.
10. Ezzi, S. A.; Urusbitani, M.; Julien, J. P. Wild-type superoxide dismutase acquires binding and toxic properties of ALS-linked mutant forms through oxidation. Journal of Neurochemistry 2007, 102, 170-178, doi:10.1111/j.1471-4159.2007.04531.x.
11. Trist, B. G.; Genoud, S.; Roudeau, S.; Rookyard, A.; Abdeen, A.; Cottam, V.; Hare, D. J.; White, M.; Altvater, J.; Fifita, J. A.; et al. Altered SOD1 maturation and post-translational modification in amyotrophic lateral sclerosis spinal cord. Brain 2022, doi:10.1093/brain/awac165.
12. Mueller, C.; Berry, J. D.; McKenna-Yasek, D. M.; Gernoux, G.; Owegi, M. A.; Pothier, L. M.; Douthwright, C. L.; Gelevski, D.; Luppino, S. D.; Blackwood, M.; et al. SOD1 Suppression with Adeno-Associated Virus and MicroRNA in Familial ALS. N Engl J Med 2020, 383, 151-158, doi:10.1056/NEJMoa2005056.
13. Miller, T.; Cudkowicz, M.; Shaw, P. J.; Andersen, P. M.; Atassi, N.; Bucelli, R. C.; Genge, A.; Glass, J.; Ladha, S.; Ludolph, A. L., et al. Phase 1-2 Trial of Antisense Oligonucleotide Tofersen for SOD1 ALS. N Engl J Med 2020, 383, 109-119, doi:10.1056/NEJMoa2003715.
14. Boros, B. D.; Schoch, K. M.; Kreple, C. J.; Miller, T. M. Antisense Oligonucleotides for the Study and Treatment of ALS. Neurotherapeutics 2022, doi:10.1007/s13311-022-01247-2.
15. van Blitterswijk, M.; Gulati, S.; Smoot, E.; Jaffa, M.; Maher, N.; Hyman, B. T.; Ivinson, A. J.; Scherzer, C. R.; Schoenfeld, D. A.; Cudkowicz, M. E.; et al. Anti-superoxide dismutase antibodies are associated with survival in patients with sporadic amyotrophic lateral sclerosis. Amyotroph Lateral Scler 2011, 12, 430-438, doi:10.3109/17482968.2011.585163.
16. Elchuri, S.; Oberley, T. D.; Qi, W.; Eisenstein, R. S.; Jackson Roberts, L.; Van Remmen, H.; Epstein, C. J.; Huang, T. T. CuZnSOD deficiency leads to persistent and widespread oxidative damage and hepatocarcinogenesis later in life. Oncogene 2005, 24, 367-380, doi:10.1038/sj.onc.1208207.
17. Kondo, T.; Reaume, A. G.; Huang, T. T.; Carlson, E.; Murakami, K.; Chen, S. F.; Hoffman, E. K.; Scott, R. W.; Epstein, C. J.; Chan, P. H. Reduction of CuZn-superoxide dismutase activity exacerbates neuronal cell injury and edema formation after transient focal cerebral ischemia. J Neurosci 1997, 17, 4180-4189.
18. Reaume, A. G.; Elliott, J. L.; Hoffman, E. K.; Kowall, N. W.; Ferrante, R. J.; Siwek, D. F.; Wilcox, H. M.; Flood, D. G.; Beal, M. F.; Brown, R. H., Jr.; et al. Motor neurons in Cu/Zn superoxide dismutase-deficient mice develop normally but exhibit enhanced cell death after axonal injury. Nat Genet 1996, 13, 43-47, doi:10.1038/ng0596-43.
19. Shefner, J. M.; Reaume, A. G.; Flood, D. G.; Scott, R. W.; Kowall, N. W.; Ferrante, R. J.; Siwek, D. F.; Upton-Rice, M.; Brown, R. H., Jr. Mice lacking cytosolic copper/zinc superoxide dismutase display a distinctive motor axonopathy. Neurology 1999, 53, 1239-1246, doi:10.1212/wnl.53.6.1239.
20. Shibata, N.; Asayama, K.; Hirano, A.; Kobayashi, M. Immunohistochemical study on superoxide dismutases in spinal cords from autopsied patients with amyotrophic lateral sclerosis. Dev Neurosci 1996, 18, 492-498, doi:10.1159/000111445.
21. Jonsson, P. A.; Emhill, K.; Andersen, P. M.; Bergemalm, D.; Brannstrom, T.; Gredal, O.; Nilsson, P.; Marklund, S. L. Minute quantities of misfolded mutant superoxide dismutase-1 cause amyotrophic lateral sclerosis. Brain 2004, 127, 73-88, doi:10.1093/brain/awh005.
22. Bruijn, L. I.; Houseweart, M. K.; Kato, S.; Anderson, K. L.; Anderson, S. D.; Ohama, E.; Reaume, A. G.; Scott, R. W.; Cleveland, D. W. Aggregation and motor neuron toxicity of an ALS-linked SOD1 mutant independent from wild-type SOD1. Science 1998, 281, 1851-1854, doi:10.1126/science.281.5384.1851.
23. Zhong, Y., Wang, J.; Henderson, M. J., Yang, P.; Hagen, B. M.; Siddique, T.; Vogel, B. E.; Deng, H. X.; Fang, S. Nuclear export of misfolded SOD1 mediated by a normally buried NES-like sequence reduces proteotoxicity in the nucleus. Elife 2017, 6, doi:10.7554/eLife.23759.
24. Saccon, R. A.; Bunton-Stasyshyn, R. K. A.; Fisher, E. M. C.; Fratta, P. Is SOD1 loss of function involved in amyotrophic lateral sclerosis? Brain: a journal of neurology 2013, 136, 2342-2358, doi:10.1093/brain/awt097.
25. Trist, B. G.; Davies, K. M.; Cottam, V.; Genoud, S.; Ortega, R.; Roudeau, S.; Carmona, A.; De Silva, K.; Wasinger, V.; Lewis, S. J. G.; et al. Amyotrophic lateral sclerosis-like superoxide dismutase 1 proteinopathy is associated with neuronal loss in Parkinson's disease brain. Acta Neuropathol 2017, 134, 113-127, doi:10.1007/s00401-017-1726-6.
26. Takeuchi, S.; Fujiwara, N.; Ido, A.; Oono, M.; Takeuchi, Y.; Tateno, M.; Suzuki, K.; Takahashi, R.; Tooyama, I.; Taniguchi, N.; et al. Induction of protective immunity by vaccination with wild-type apo superoxide dismutase 1 in mutant SOD1 transgenic mice. J Neuropathol Exp Neurol 2010, 69, 1044-1056, doi:10.1097/NEN.0b013e3181f4a90a.
27. Urushitani, M.; Ezzi, S. A.; Julien, J.-P. Therapeutic effects of immunization with mutant superoxide dismutase in mice models of amyotrophic lateral sclerosis. Proceedings of the National Academy of Sciences of the United States of America 2007, 104, 2495-2500, doi:10.1073/pnas.0606201104.
28. Liu, H. N.; Tjostheim, S.; Dasilva, K.; Taylor, D.; Zhao, B.; Rakhit, R.; Brown, M.; Chakrabartty, A.; McLaurin, J.; Robertson, J. Targeting of monomer/misfolded SOD1 as a therapeutic strategy for amyotrophic lateral sclerosis. J Neurosci 2012, 32, 8791-8799, doi:10.1523/JNEUROSCI.5053-11.2012.
29. Gros-Louis, F.; Soucy, G.; Lariviere, R.; Julien, J. P. Intracerebroventricular infusion of monoclonal antibody or its derived Fab fragment against misfolded forms of SOD1 mutant delays mortality in a mouse model of ALS. J Neurochem 2010, 113, 1188-1199, doi:10.1111/j.1471-4159.2010.06683.x.
30. Maier, M.; Welt, T.; Wirth, F.; Montrasio, F.; Preisig, D.; McAfoose, J.; Vieira, F. G.; Kulic, L.; Späni, C.; Stehle, T.; et al. A human-derived antibody targets misfolded SOD1 and ameliorates motor symptoms in mouse models of amyotrophic lateral sclerosis. Science translational medicine 2018, 10, eaah3924, doi:10.1126/scitranslmed.aah3924.
31. Silva-Pilipich, N.; Smerdou, C.; Vanrell, L. A Small Virus to Deliver Small Antibodies: New Targeted Therapies Based on AAV Delivery of Nanobodies. Microorganisms 2021, 9, doi:10.3390/microorganisms9091956.
32. Ghadge, G. D.; Kay, B. K.; Drigotas, C.; Roos, R. P. Single chain variable fragment antibodies directed against SOD1 ameliorate disease in mutant SOD1 transgenic mice. Neurobiol Dis 2019, 121, 131-137, doi:10.1016/j.nbd.2018.08.021.
33. Patel, P.; Kriz, J.; Gravel, M.; Soucy, G.; Bareil, C.; Gravel, C.; Julien, J. P. Adeno-associated virus-mediated delivery of a recombinant single-chain antibody against misfolded superoxide dismutase for treatment of amyotrophic lateral sclerosis. Molecular Therapy 2014, 22, 498-510, doi:10.1038/mt.2013.239.
34. Muyldermans, S. A guide to: generation and design of nanobodies. FEBS J 2021, 288, 2084-2102, doi:10.1111/febs.15515.
35. Chatterjee, D.; Bhatt, M.; Butler, D.; De Genst, E.; Dobson, C. M.; Messer, A.; Kordower, J. H. Proteasome-targeted nanobodies alleviate pathology and functional decline in an alpha-synuclein-based Parkinson's disease model. NPJ Parkinsons Dis 2018, 4, 25, doi:10.1038/s41531-018-0062-4
36. Pothin, E.; Lesuisse, D., Lafaye, P. Brain Delivery of Single-Domain Antibodies: A Focus on VHH and VNAR. Pharmaceutics 2020, 12, doi:10.3390/pharmaceutics12100937.
37. Danis, C.; Dupre, E.; Zejneli, O.; Caillierez, R.; Arrial, A.; Begard, S.; Mortelecque, J.; Eddarkaoui, S.; Loyens, A.; Cantrelle, F. X.; et al. Inhibition of Tau seeding by targeting Tau nucleation core within neurons with a single domain antibody fragment. Mol Ther 2022, 30, 1484-1499, doi:10.1016/j.ymthe.2022.01.009.
38. Bosco, D. A.; Morfini, G.; Karabacak, N. M.; Song, Y.; Gros-Louis, F.; Pasinelli, P.; Goolsby, H.; Fontaine, B. A.; Lemay, N.; Mckenna-Yasek, D.; et al. Wild-type and mutant SOD1 share an aberrant conformation and a common pathogenic pathway in ALS. Nature Neuroscience 2010, 13, 1396-1403, doi:10.1038/nn.2660.
39. Deng, H. X.; Hentati, A.; Tainer, J. A.; Iqbal, Z.; Cayabyab, A.; Hung, W. Y.; Getzoff, E. D.; Hu, P.; Herzfeldt, B.; Roos, R. P.; et al. Amyotrophic lateral sclerosis and structural defects in Cu,Zn superoxide dismutase. Science 1993, 261, 1047-1051, doi:10.1126/science.8351519.
40. Molnar, K. S.; Karabacak, N. M.; Johnson, J. L.; Wang, Q.; Tiwari, A.; Hayward, L. J.; Coales, S. J.; Hamuro, Y.; Agar, J. N. A common property of amyotrophic lateral sclerosis-associated variants: destabilization of the copper/zinc superoxide dismutase electrostatic loop. The Journal of biological chemistry 2009, 284, 30965-30973, doi:10.1074/jbc.M109.023945.
41. Rechsteiner, M.; Rogers, S. W. PEST sequences and regulation by proteolysis. Trends Biochem Sci 1996, 21, 267-271.
42. Butler, D. C.; Joshi, S. N.; Genst, E.; Baghel, A. S.; Dobson, C. M.; Messer, A. Bifunctional Anti-Non-Amyloid Component alpha-Synuclein Nanobodies Are Protective In Situ. PLOS One 2016, 11, e0165964, doi:10.1371/journal.pone.0165964.
43. Joshi, S. N.; Butler, D. C.; Messer, A. Fusion to a highly charged proteasomal retargeting sequence increases soluble cytoplasmic expression and efficacy of diverse anti-synuclein intrabodies. MAbs 2012, 4, 686-693, doi:10.4161/mabs.21696.
44. Butler, D. C.; Messer, A. Bifunctional anti-huntingtin proteasome-directed intrabodies mediate efficient degradation of mutant huntingtin exon 1 protein fragments. PLOS One 2011, 6, e29199, doi:10.1371/journal.pone.0029199.
45. Crapo, J. D.; Oury, T.; Rabouille, C.; Slot, J. W.; Chang, L. Y. Copper,zinc superoxide dismutase is primarily a cytosolic protein in human cells. Proc Natl Acad Sci USA 1992, 89, 10405-10409, doi:10.1073/pnas.89.21.10405.
46. Rodriguez, J. A.; Valentine, J. S.; Eggers, D. K.; Roe, J. A.; Tiwari, A.; Brown, R. H.; Hayward, L. J. Familial amyotrophic lateral sclerosis-associated mutations decrease the thermal stability of distinctly metallated species of human copper/zinc superoxide dismutase. The Journal of biological chemistry 2002, 277, 15932-15937, doi:10.1074/jbc.M112088200.
47. Borchelt, D. R.; Lee, M. K.; Slunt, H. S.; Guamieri, M.; Xu, Z. S.; Wong, P. C.; Brown, R. H., Jr.; Price, D. L.; Sisodia, S. S.; Cleveland, D. W. Superoxide dismutase 1 with mutations linked to familial amyotrophic lateral sclerosis possesses significant activity. Proc Natl Acad Sci USA 1994, 91, 8292-8296, doi:10.1073/pnas.91.17.8292.
48. Boopathy, S.; Silvas, T. V.; Tischbein, M.; Jansen, S.; Shandilya, S. M.; Zitzewitz, J. A.; Landers, J. E.; Goode, B. L.; Schiffer, C. A.; Bosco, D. A. Structural basis for mutation-induced destabilization of profilin 1 in ALS. Proceedings of the National Academy of Sciences 2015, 112, 7984-7989, doi:10.1073/pnas. 1424108112.
49. Auclair, J. R.; Boggio, K. J.; Petsko, G. A.; Ringe, D.; Agar, J. N. Strategies for stabilizing superoxide dismutase (SOD1), the protein destabilized in the most common form of familial amyotrophic lateral sclerosis. Proc Natl Acad Sci USA 2010, 107, 21394-21399, doi:10.1073/pnas.1015463107.
50. Gao, K.; Oerlemans, R.; Groves, M. R. Theory and applications of differential scanning fluorimetry in early-stage drug discovery. Biophys Rev 2020, 12, 85-104, doi:10.1007/s12551-020-00619-2.
51. Niesen, F. H.; Berglund, H.; Vedadi, M. The use of differential scanning fluorimetry to detect ligand interactions that promote protein stability. Nat Protoc 2007, 2, 2212-2221, doi:10.1038/nprot.2007.321.
52. Chen, H.; Qian, K.; Du, Z.; Cao, J.; Petersen, A.; Liu, H.; Blackbourn, L. W. t.; Huang, C. L.; Errigo, A.; Yin, Y.; et al. Modeling ALS with iPSCs reveals that mutant SOD1 misregulates neurofilament balance in motor neurons. Cell Stem Cell 2014, 14, 796-809, doi:10.1016/j.stem.2014.02.004.
53. Kiskinis, E.; Sandoe, J.; Williams, L. A.; Boulting, G. L.; Moccia, R.; Wainger, B. J.; Han, S.; Peng, T.; Thams, S.; Mikkilineni, S.; et al. Pathways disrupted in human ALS motor neurons identified through genetic correction of mutant SOD1. Cell Stem Cell 2014, 14, 781-795, doi:10.1016/j.stem.2014.03.004.
54. Rigamonti, A.; Repetti, G. G.; Sun, C.; Price, F. D.; Reny, D. C.; Rapino, F.; Weisinger, K.; Benkler, C.; Peterson, Q. P.; Davidow, L. S.; et al. Large-Scale Production of Mature Neurons from Human Pluripotent Stem Cells in a Three-Dimensional Suspension Culture System. Stem Cell Reports 2016, 6, 993-1008, doi:10.1016/j.stemcr.2016.05.010.
55. Robberecht, W. I. M.; Rousseau, F.; Schymkowitz, J. Sinlge domain antibodies against sod 1 and their use in medicine. WO 2014/191493 A1, 2014 May 28, 2014.
56. Shvil, N.; Banerjee, V.; Zoltsman, G.; Shani, T.; Kahn, J.; Abu-Hamad, S.; Papo, N.; Engel, S.; Bernhagen, J., Israelson, A. MIF inhibits the formation and toxicity of misfolded SOD1 amyloid aggregates: implications for familial ALS. Cell Death Dis 2018, 9, 107, doi:10.1038/s41419-017-0130-4.
57. Sau, D.; De Biasi, S.; Vitellaro-Zuccarello, L.; Riso, P.; Guarnieri, S.; Porrini, M.; Simeoni, S.; Crippa, V.; Onesto, E.; Palazzolo, I.; et al. Mutation of SOD1 in ALS: a gain of a loss of function. Hum Mol Genet 2007, 16, 1604-1618, doi:10.1093/hmg/ddm110.
58. Trist, B. G.; Hare, D. J.; Double, K. L. A Proposed Mechanism for Neurodegeneration in Movement Disorders Characterized by Metal Dyshomeostasis and Oxidative Stress. Cell Chem Biol 2018, 25, 807-816, doi:10.1016/j.chembiol.2018.05.004.
59. Hossain, M. A.; Sarin, R.; Donnelly, D. P.; Miller, B. C.; Salisbury, J. P.; Conway, J. B.; Watson, S.; Winters, J. N.; Alam, N.; Sivasankar, D.; et al. Protein crosslinking as a therapeutic strategy for SOD1-related ALS. bioRxiv 2021, 2021.2006.2023.449516, doi:10.1101/2021.06.23.449516.
60. Roberts, B. R.; Lim, N. K.; McAllum, E. J.; Donnelly, P. S.; Hare, D. J.; Doble, P. A.; Turner, B. J.; Price, K. A.; Lim, S. C.; Paterson, B. M.; et al. Oral treatment with Cu(II)(atsm) increases mutant SOD1 in vivo but protects motor neurons and improves the phenotype of a transgenic mouse model of amyotrophic lateral sclerosis. J Neurosci 2014, 34, 8021-8031, doi:10.1523/JNEUROSCI.4196-13.2014.
61. Hilton, J. B.; Mercer, S. W.; Lim, N. K.; Faux, N. G.; Buncic, G.; Beckman, J. S.; Roberts, B. R.; Donnelly, P. S.; White, A. R.; Crouch, P. J. Cu(II)(atsm) improves the neurological phenotype and survival of SOD1(G93A) mice and selectively increases enzymatically active SOD1 in the spinal cord. Sci Rep 2017, 7, 42292, doi:10.1038/srep42292
62. Nikseresht, S.; Hilton, J. B. W.; Kysenius, K.; Liddell, J. R.; Crouch, P. J. Copper-ATSM as a Treatment for ALS: Support from Mutant SOD1 Models and Beyond. Life (Basel) 2020, 10, doi:10.3390/life 10110271.
63. Abel, O.; Powell, J. F.; Andersen, P. M.; Al-Chalabi, A. ALSOD: A user-friendly online bioinformatics tool for amyotrophic lateral sclerosis genetics. Hum Mutat 2012, 33, 1345-1351, doi:10.1002/humu.22157.
64. Pleiner, T.; Bates, M.; Trakhanov, S.; Lee, C. T.; Schliep, J. E.; Chug, H.; Böhning, M.; Stark, H.; Urlaub, H.; Görlich, D. Nanobodies: Site-specific labeling for super-resolution imaging, rapid epitope-mapping and native protein complex isolation. eLife 2015, 4, 1-21, doi:10.7554/eLife.11349.
65. Frey, S.; Gorlich, D. A new set of highly efficient, tag-cleaving proteases for purifying recombinant proteins. J Chromatogr A 2014, 1337, 95-105, doi:10.1016/j.chroma.2014.02.029.
66. Baron, D. M.; Kaushansky, L. J.; Ward, C. L.; Sama, R. R. K.; Chian, R.-J.; Boggio, K. J.; Quaresma, A. J. C.; Nickerson, J. A.; Bosco, D. A. Amyotrophic lateral sclerosis-linked FUS/TLS alters stress granule assembly and dynamics. Molecular neurodegeneration 2013, 8, 30, doi:10.1186/1750-1326-8-30.
67. Lin, Y. C.; Kumar, M. S.; Ramesh, N.; Anderson, E. N.; Nguyen, A. T.; Kim, B.; Cheung, S.; McDonough, J. A.; Skarnes, W. C.; Lopez-Gonzalez, R.; et al. Interactions between ALS-linked FUS and nucleoporins are associated with defects in the nucleocytoplasmic transport pathway. Nat Neurosci 2021, 24, 1077-1088, doi:10.1038/s41593-021-00859-9.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A single-domain antibody that binds to Superoxide dismutase 1 (SOD1) comprising an amino acid sequence that is at least 80% identical to of any one of SEQ ID NO: 2-24.

2. The single-domain antibody according to claim 1, wherein SOD1 is human SOD1.

3. The single-domain antibody according to claim 1, wherein the single-domain antibody binds mutant SOD1.

4. The single-domain antibody according to claim 3, wherein the mutant SOD1 is characterized by a mutation of amino acids at positions 4 and/or 93.

5. The single-domain antibody of claim 1, wherein the single-domain antibody binds both wild-type and mutant SOD1.

6. A fusion protein comprising the single-domain antibody of claim 1, fused to a tag.

7. The fusion protein of claim 6, wherein the tag is a degradation tag.

8. The fusion protein of claim 6, wherein the degradation tag is PEST.

9. A nucleic acid molecule encoding the single-domain antibody of claim 1.

10. A vector comprising the nucleic acid molecule according to claim 10.

11. A host cell comprising the nucleic acid molecule according to claim 10.

12. A method of diagnosing and monitoring a motor neuron pathology the method comprising: administering the single-domain antibody of claim 1 to a subject.

13. A method of treating a motor neuron pathology or improving symptoms of the motor neuron pathology in a subject, the method comprising: administering a therapeutically effective amount of the single-domain antibody of claim 1 to the subject.

14. The method according to claim 12 or 13, wherein the motor neuron pathology is amyotrophic lateral sclerosis (ALS).

15. The method according to claim 12 or 13, wherein the single-domain antibody is administered intracerebroventricularly.

16. The method according to claim 12 or 13, wherein the single-domain antibody is administered through gene therapy.

17. The method of any one of claims 12-16, wherein the method further comprises administering a neurotrophin.

18. A medicament comprising: the single-domain antibody of claim 1, and a pharmaceutically acceptable excipient.
